(19) United States
(12) Patent Application Publication
(10) Pub. No.: US 2022/0212156 A1
(43) Pub. Date: Jul. 7, 2022

(54) MICROCAPSULE

(71) Applicants: COMMONWEALTH SCIENTIFIC AND INDUSTRIAL RESEARCH ORGANISATION, Acton (AU); THE UNIVERSITY OF QUEENSLAND, Queensland (AU)

(72) Inventors: Alison Louise White, Margate (AU); Simon Richard Biggs, Perth (AU); Hazel Anne Javier, Archerfield (AU)

(21) Appl. No.: 17/595,165

(22) PCT Filed: May 8, 2020
(86) PCT No.: PCT/AU2020/050461

§ 371 Date: Nov. 10, 2021

(30) Foreign Application Priority Data
May 10, 2019 (AU) 2019901608

(51) Int. Cl. B01J 13/22; B01J 13/10; B01J 13/08; C05G 5/35; C05B 13/06; A61K 9/50; A61K 8/11; A61Q 19/00; A01N 25/28

(52) U.S. Cl. CPC B01J 13/22; B01J 13/10; B01J 13/08; C05G 5/35; A01N 25/28; A61K 9/501; A61K 9/5026; A61K 8/11; A61Q 19/00; C05B 13/06

(57) ABSTRACT

The present disclosure relates to electroless plating process for preparing microcapsules comprising an ionic shell encapsulating a fluid core. Platinum nanoparticles are used as the catalyst for the electroless plating process and are found in the fluid core. The ionic shell is comprised of inorganic salts, such as salts of alkaline earth metals. In particular electroless plating of calcium phosphate shell on to fluid cores, polymer encapsulated fluid cores and gel cores are disclosed.

### FIELD

The present disclosure generally relates to microcapsules, processes for preparing microcapsules, and applications for the microcapsules.

### BACKGROUND

Retention of soluble and volatile chemicals within microcapsules has been a significant challenge for decades. Encapsulation of an active has two key roles: i) it protects the active ingredient (AI) from the external environment, and ii) it allows for a controlled release of the AI.

Conventionally, organic polymers have been the preferred material for microcapsule shells. However, the inherent porous nature of such polymer shells makes them generally unsuitable for retaining small molecules, which can diffuse through the polymer matrix and be lost to the environment. Improving core retention of polymer microcapsules by modifying the properties of the polymer shell has had limited success. More recently focus has been directed to developing novel core-shell microcapsules that provide retention of low molecular weight volatile molecules until sufficient force is applied to trigger release, for example by electroless deposition of a thin metal shell onto polymer microcapsules. However, whilst metal shells have shown to provide good retention properties, they are an expensive option compared to polymer shell microcapsules.

In order to make the microcapsules attractive and affordable: i) the shell should be suitably thin so that it can easily be broken when required, whilst still retaining its impermeable nature pre-breakage; and, ii) the material chosen for the shell should be cost-effective to allow for use in industrial products such as paints, pesticides, insect repellents, sunscreen, fragrances, laundry detergents, agrochemicals and nutraceuticals.

Consequently, there is a need to provide alternative or improved microcapsules and processes for preparing alternative or improved microcapsules.

### SUMMARY

The present inventors have prepared a microcapsule comprising an ionic shell. The ionic shell may be an inorganic calcium phosphate shell. The microcapsule may comprise an inorganic calcium phosphate shell encapsulating an inner fluid core. The inner fluid core may be selected from a liquid or a gel. In an embodiment or example, the inner liquid core may comprises platinum nanoparticles, for example as stabilised platinum nanoparticles to support encapsulation by the ionic shell. The microcapsules may be delivered in a targeted manner or in response to a specific trigger. The present inventors have also identified a process for preparing microcapsules comprising an ionic shell such as an inorganic calcium phosphate shell. The process can comprise providing an inner fluid core comprising platinum nanoparticles, and encapsulating the inner liquid core with an ionic shell, for example an inorganic calcium phosphate shell.

The present inventors have surprisingly found that presence of platinum nanoparticles in the fluid core material can promote growth of an ionic shell, for example an inorganic calcium phosphate shell, on the surface of a microcapsule. One or more advantages of the present disclosure according to at least some embodiments or examples as described herein is that the platinum nanoparticles can produce microcapsules that are substantially impermeable to low molecular weight volatile molecules encapsulated therein until release of the encapsulated molecules is desired.

In one aspect there is provided a microcapsule comprising an ionic shell encapsulating a fluid core, wherein the fluid core comprises platinum nanoparticles.

In an embodiment, the ionic shell may be an inorganic calcium phosphate shell. The inorganic calcium phosphate shell may comprise one or more calcium phosphate compounds selected from monocalcium phosphate, dicalcium phosphate, tricalcium phosphate, octacalcium phosphate, dicalcium diphosphate, calcium triphosphate, calcium hydroxide phosphate, or combinations thereof.

In another embodiment, the fluid core may be selected from a liquid or a gel. In another embodiment, the platinum nanoparticles may be polymer stabilised platinum nanoparticles. The platinum nanoparticles may be prepared from one or more platinum compounds selected from PtCl₂, H₂PtCl₆, K₂PtCl₆, Na₂PtCl₆, K₂PtCl₄, or combinations thereof. The diameter of the platinum nanoparticles may between about 0.1 nm and 200 nm.

In another embodiment, the ionic shell may have a thickness of between about 1 nm to about 1000 nm. In one example, the ionic shell is impermeable to molecules smaller than 500 g·mol⁻¹.

In another embodiment, the fluid core may comprise or consist of a fluid carrier comprising one or more active agents and platinum nanoparticles according to any examples or embodiments as described herein.

In an embodiment or example, the active agent may be a water soluble active agent or an oil soluble active.

In another embodiment, the fluid core may comprise or further optionally consist of one or more additives selected from an oil carrier, an aqueous carrier, a solid, a water/oil emulsion, and a contrast agent. The fluid core may comprise between about 45% to about 99.9% by weight of the microcapsule.

In another embodiment, the fluid core may comprise or further optionally consist of an inner coating encapsulating the fluid core from the ionic shell, wherein the ionic shell encapsulates the inner coating. In another example, the inner coating is a polymeric shell. The polymeric shell may be selected from a synthetic polymer or a naturally-occurring polymer.

In an embodiment, the inner coating may have a thickness of between about 10 nm to about 5000 nm. In another embodiment, the ratio by weight of the fluid core to inner coating is between about 6:1 to 1:1.

In an embodiment, the platinum nanoparticles may be present in the fluid core or inner coating for catalysing an electroless plating deposition of the outer ionic shell thereon.

In another embodiment, the platinum nanoparticles may be present in the fluid core at the interface of the inner fluid core as a Pickering stabiliser. In yet another embodiment, the platinum nanoparticles may be provided in the inner coating as a Pickering stabiliser or at a solid-aqueous interface between the inner fluid core and inner coating.

In an embodiment, the diameter of the microcapsules may be between about 0.05 μm to about 1000 μm.

In another aspect, there is provided a composition comprising a plurality of microcapsules defined above or according to any embodiments or examples thereof as described herein.

In another aspect, there is provided a process for preparing a microcapsule, the process comprising providing an inner fluid core comprising platinum nanoparticles, and encapsulating the fluid liquid core with an ionic shell, for example an inorganic calcium phosphate shell. The ionic shell may be provided or deposited as a densely packed layer of calcium phosphate compounds over the fluid core by electroless plating catalysed by the platinum nanoparticles present at the surface of the fluid core. In an embodiment, the platinum nanoparticles may be at least embedded within the fluid core prior to deposition of the ionic shell. The fluid core may a liquid or a gel.

In another embodiment, the process may further comprise encapsulating the fluid core by an inner polymeric coating using an emulsification process. In an example, the platinum nanoparticles may be adsorbed on, at or near the surface of the inner coating to form a discontinuous layer during the emulsification process. In another example, the platinum nanoparticles may be embedded in or on the surface of the inner coating to form a discontinuous layer during the emulsification process.

In another aspect, there is provided a use of the microcapsule defined above or according to any embodiments or examples thereof as described herein, or composition comprising a plurality of the microcapsules defined above or according to any embodiments or examples thereof as described herein, as a drug delivery vehicle or carrier in controlled release of the inner fluid core comprising an active agent.

In an embodiment, the microcapsule according to at least some examples as described herein may be used as an implant within a subject for controlled release of the active agent to a subject.

In another embodiment, the controlled release may be a sustained release, for example capable of being used to provide systemically administered doses.

In another aspect, there is provided a use of the microcapsule defined above or according to any embodiments or examples thereof as described herein, or composition comprising or consisting a plurality of the microcapsules defined above or according to any embodiments or examples thereof as described herein, in personal care products.

In another aspect, there is provided a use of the microcapsule defined above or according to any embodiments or examples thereof as described herein, or composition comprising or consisting a plurality of the microcapsules defined above or according to any embodiments or examples thereof as described herein, in agricultural products.

In another aspect, there is provided a use of the microcapsule defined above or according to any embodiments or examples thereof as described herein, or composition comprising or consisting a plurality of the microcapsules defined above or according to any embodiments or examples thereof as described herein, in food products.

In another embodiment of any of the above aspects, embodiments or examples, the release of the inner fluid core comprising or consisting of the active agent may be activated by ultrasound, degradation, or mechanical fracture.

### BRIEF DESCRIPTION OF THE DRAWINGS

Some embodiments of the present disclosure are described and illustrated herein, by way of example only, with reference to the accompanying Figures in which:

FIG. 1a is a scanning electron micrograph showing a) calcium phosphate coated microcapsules with a shell thickness of 30 nm achieved with 96 mM calcium chloride reagent concentration, and b) calcium phosphate coated microcapsules with a shell thickness of 50 nm achieved with 192 mM calcium chloride reagent concentration.

FIG. 1b is a scanning electron micrograph showing a) complete calcium phosphate shells where CaCl₂) Na₂H₂PO₄ molar ratio was 2.1:1, b) partial calcium phosphate coatings where CaCl₂:Na₂H₂PO₄ molar ratio was 4.4:1 at 60° C. for 15 min. and c) shows a representative energy-dispersive x-ray spectrum confirming the shell to contain calcium and phosphorus.

FIG. 2 is scanning electron micrographs of calcium phosphate shell formation where the electroless deposition was allowed to continue at room temperature for (a) no additional time (b) 2 hours (c) 4 hours and (d) 6 hours without stirring, after the initial 15 minutes at 60° C.

FIG. 3 is a ¹⁹F Nuclear Magnetic Resonance spectra to detect release of PFOB from the microcapsules for (a) PLGA capsules with no calcium phosphate shell, b) PLGA microcapsules with a partial calcium phosphate shell and c) PLGA microcapsules with a complete calcium phosphate shell (molar ratio CaCl₂:NaH₂PO₂ of 2.1:1). Perfluorobenzoic acid (pFBA) was used as an internal standard. All samples were dispersed in deuterated chloroform for 2 weeks at 40° C. before the spectra were acquired.

FIG. 4 is a plot of % release of hexyl salicylate from the microcapsules, into 80:20 ethanol:water, as measured by gas chromatography from PLGA capsules with no calcium phosphate shell (triangles), PLGA capsules with a porous calcium phosphate shell (with NaF) (squares) and PLGA capsules with a complete calcium phosphate shell (without NaF) (diamonds). B) and C) show scanning electron micrographs of PLGA capsules with a porous calcium phosphate shell and PLGA capsules with a complete calcium phosphate shell respectively.

FIG. 5 is a) calibration curve, b) mass spectrum showing m/z 223.23 peak corresponding to hexyl salicylate, and c) plot of % release of hexyl salicylate from PLGA only microcapsules compared to calcium phosphate coated microcapsules prepared at 60° C. for 15 minutes and left to stand for 6 hours at room temperature.

FIG. 6 is scanning electron micrographs showing complete calcium phosphate shell deposited onto calcium alginate microcapsule beads with PVP-PT-NPs adsorbed, three magnifications of surface indicated by box on first image, and elemental analysis confirming presence of Ca and P elements.

FIG. 7 is a plot showing release of toluene from (top) uncoated alginate microcapsule beads and (bottom) alginate microcapsule beads with a calcium phosphate ionic shell where the absorbance peak at 9.58 min corresponds to toluene measured at 265 nm wavelength.

### DETAILED DESCRIPTION

The present disclosure describes the following various non-limiting examples, which relate to investigations undertaken to identify alternative and improved microcapsules and processes for preparing the microcapsules. The present inventors have prepared a microcapsule comprising an ionic shell. The microcapsule can comprise an ionic shell encapsulating a fluid core comprising platinum nanoparticles. The ionic shell can be an inorganic calcium phosphate shell. The fluid core can comprise stabilised platinum nanoparticles. In another example, the microcapsules comprise an inner coating encapsulating a fluid core, and an ionic shell encapsulating the inner coating. The present inventors have also identified a process for preparing the microcapsules wherein an inner fluid core composition comprises platinum nanoparticles.

At least according to some embodiments or examples as described herein, the present disclosure provides an alternative or improved microcapsule that has been prepared by depositing an outer ionic shell using platinum nanoparticles as a catalyst, under fast, mild conditions, which delivers improved impermeable characteristics to the microcapsules, in a more cost-effective manner.

### GENERAL TERMS

Throughout this disclosure, unless specifically stated otherwise or the context requires otherwise, reference to a single step, composition of matter, group of steps or group of compositions of matter shall be taken to encompass one and a plurality (i.e. one or more) of those steps, compositions or matter, groups of steps or groups of composition of matter. Thus, as used herein, the singular forms “a”, “an” and “the” include plural aspects unless the context clearly indicates otherwise. For example, reference to “a” includes a single as well as two or more; reference to “an” includes a single as well as two or more; reference to “the” includes a single as well as two or more and so forth.

Those skilled in the art will appreciate that the disclosure herein is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

Each example of the present disclosure described herein is to be applied mutatis mutandis to each and every other example unless specifically stated otherwise. The present disclosure is not to be limited in scope by the specific examples described herein, which are intended for the purpose of exemplification only. Functionally-equivalent products, compositions and methods are clearly within the scope of the disclosure as described herein.

The term “and/or”, e.g., “X and/or Y” shall be understood to mean either “X and Y” or “X or Y” and shall be taken to provide explicit support for both meanings or for either meaning.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The term “consists of”, or variations such as “consisting of”, refers to the inclusion of any stated element, integer or step, or group of elements, integers or steps, that are recited in context with this term, and excludes any other element, integer or step, or group of elements, integers or steps, that are not recited in context with this term.

Unless otherwise indicated, the terms “first,” “second,” etc. are used herein merely as labels, and are not intended to impose ordinal, positional, or hierarchical requirements on the items to which these terms refer. Moreover, reference to a “second” item does not require or preclude the existence of lower-numbered item (e.g., a “first” item) and/or a higher-numbered item (e.g., a “third” item).

As used herein, the phrase “at least one of”, when used with a list of items, means different combinations of one or more of the listed items may be used and only one of the items in the list may be needed. The item may be a particular object, thing, or category. In other words, “at least one of” means any combination of items or number of items may be used from the list, but not all of the items in the list may be required. For example, “at least one of item A, item B, and item C” may mean item A; item A and item B; item B; item A, item B, and item C; or item B and item C. In some cases, “at least one of item A, item B, and item C” may mean, for example and without limitation, two of item A, one of item B, and ten of item C; four of item B and seven of item C; or some other suitable combination.

Reference herein to “example,” “one example,” “another example,” or similar language means that one or more feature, structure, element, component or characteristic described in connection with the example is included in at least one embodiment or implementation. Thus, the phrases “in one example,” “as one example,” and similar language throughout the present disclosure may, but do not necessarily, refer to the same example. Further, the subject matter characterizing any one example may, but does not necessarily, include the subject matter characterizing any other example.

It will be clearly understood that, although a number of prior art publications are referred to herein, this reference does not constitute an admission that any of these documents forms part of the common general knowledge in the art, in Australia or in any other country.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

### Microcapsule Composition and Structure

The present disclosure provides a microcapsule comprising an ionic shell encapsulating a fluid core. The fluid core can be a liquid or a gel. The fluid core may be a liquid core. The fluid core may be a gel core. The ionic shell may comprise or consist of an ionic compound, for example calcium phosphate. In an example, the ionic shell is an inorganic calcium phosphate shell. The liquid core may comprise platinum nanoparticles according to any examples thereof as described herein. The platinum nanoparticles may be embedded within the inner liquid core prior to deposition of the ionic shell. The gel core may comprise platinum nanoparticles according to any examples thereof as described herein. The platinum nanoparticles may be embedded within the inner gel core prior to deposition of the ionic shell. The microcapsules may further comprise an inner coating. The inner coating may comprise platinum nanoparticles according to any examples thereof as described herein. The liquid core and the inner coating may comprise platinum nanoparticles according to any examples thereof as described herein. The gel core and the inner coating may comprise platinum nanoparticles according to any examples thereof as described herein. The platinum nanoparticles may be adsorbed or embedded in the inner coating and/or liquid core. The platinum nanoparticles may be adsorbed or embedded in the inner coating and/or gel core. A microcapsule of the present disclosure can be designed to be substantially impermeable to low molecular weight volatile molecules, for example restricting or preventing release of the volatile molecules encapsulated within the inner core until the release is intentionally activated.

The fluid core may be referred to as an inner fluid core. The liquid core may be referred to as an inner liquid core. The gel core may be referred to as an inner gel core. In one example, the ionic shell is an outer ionic shell. In another example, there is provided a microcapsule comprising or consisting of an ionic shell encapsulating a fluid core, the fluid core comprising platinum nanoparticles and optionally an inner coating. In another example, there is provided a microcapsule comprising or consisting of an inner polymeric coating encapsulating a fluid core, wherein the inner polymeric coating and/or fluid core comprises platinum nanoparticles, and wherein an ionic shell encapsulates the inner polymeric coating and fluid core. In another example, there is provided a microcapsule comprising or consisting of an ionic shell encapsulating a liquid core, the liquid core comprising platinum nanoparticles and optionally an inner coating. In another example, there is provided a microcapsule comprising or consisting of an inner polymeric coating encapsulating a liquid core, wherein the inner polymeric coating and/or liquid core comprises platinum nanoparticles, and wherein an ionic shell encapsulates the inner polymeric coating and liquid core. In yet another example, there is provided a microcapsule comprising or consisting of an ionic shell encapsulating a gel core, the gel core comprising platinum nanoparticles and optionally an inner coating. In another example, there is provided a microcapsule comprising or consisting of an inner polymeric coating encapsulating a gel core, wherein the inner polymeric coating and/or gel core comprises platinum nanoparticles, and wherein an ionic shell encapsulates the inner polymeric coating and gel core.

It will be appreciated that encapsulation of low molecular weight volatile active molecules (e.g. <500 g·mol⁻¹) for controlled release in microcapsules can be of value for a broad range of applications. It will be appreciated that a controlled release may include sustained release. For example, polymer microcapsules are often used for encapsulation, however polymer microcapsules are typically unable to retain low molecular weight volatile molecules for long periods of time (typically no longer than a few hours to a few days). Precious metals have been previously used to grow more impermeable shells around microcapsules, which can provide controlled release of the microcapsule contents by an external trigger. However, these precious metals are expensive and do not lend themselves to a large range of applications due to the significant barrier of cost of goods in manufacturing.

At least according to some embodiments or example as described herein, the microcapsules of the present disclosure can provide a more cost effective and controllable production of microcapsules that are substantially impermeable to low molecular weight volatile active molecules for sustained and/or controlled release.

It will be appreciated that the size of the microcapsules can be controlled by altering factors such as the stirring speed and the shape of the stirring blade or rotor blade of the stirrer or mixer used during a microencapsulation process, or by adjusting the reaction rate by altering the polymerisation conditions (e.g. the reaction temperature and time) for the inner polymeric coating material. In particular, the size of the microcapsules may be controlled by regulating the stirring speed, which in turn can regulate the size of the droplets of the inner fluid core material in the process.

In some embodiments or examples, the diameter of each microcapsule may be between about 0.05 μm to about 1000 μm. The diameter of the microcapsule may be in a range from about 0.06 μm to about 800 μm, about 0.07 μm to about 600 μm, about 0.08 μm to about 400 μm, or about 0.1 μm to about 100 μm. The diameter of the microcapsule may be at least 0.05 μm, at least 0.07 μm, at least 0.09 μm, at least 0.1 μm, at least 0.2 μm, at least 0.4 μm, at least 0.6 μm, at least 0.8 μm, at least 1.0 μm, at least 2.0 μm, at least 4.0 μm, at least 8.0 μm, at least 12.0 μm, at least 15.0 μm, at least 20.0 μm, at least 40.0 μm, or at least 80.0 μm. The diameter of the microcapsule may be less than 1000 μm, less than 800 μm, less than 600 μm, less than 500 μm, less than 400 μm, less than 300 μm, less than 200 μm, or less than 100 μm. The diameter of the microcapsule may be in a range provided by any two lower and/or upper values as previously described.

Advantageously, the microcapsules can be delivered in a targeted manner or in response to a specific trigger. According to at least some embodiments or examples as described herein, the microcapsules can provide a capsule that is substantially impermeable and can be advantageously suitable for use in various applications. The microcapsule can be impermeable to low molecular weight volatile molecules encapsulated within it thereby preventing release. For example, the microcapsule may be impermeable to molecules smaller than 500 g·mol⁻¹.

It will be appreciated that the present disclosure may provide a composition comprising a plurality of the microcapsules. The composition may include from 0.001% to 99%, by weight of the composition of the microcapsules. In another embodiment, the composition may include from 0.01% to 90% by weight of the composition of the microcapsules. In another embodiment, the composition may include from 0.1% to 75% by weight of the composition of the microcapsules. In another embodiment, the composition may include from 0.1% to 25% by weight of the composition of the microcapsules. In another embodiment, the composition may include from 1% to 15% by weight of the composition of the microcapsules. The composition may include a mixture of different microcapsules of the present disclosure. For example, the composition may comprise a mixture of microcapsules wherein a first microcapsule comprises a first fluid core material and a second microcapsule comprises a second fluid core material. For example, the composition may comprise a mixture of microcapsules wherein a first microcapsule comprises a first liquid core material and a second microcapsule comprises a second liquid core material. For example, the composition may comprise a mixture of microcapsules wherein a first microcapsule comprises a first liquid core material and a second microcapsule comprises a second gel core material. For example, the composition may comprise a mixture of microcapsules wherein a first microcapsule comprises a first gel core material and a second microcapsule comprises a second gel core material. It will be appreciated that the size distribution of the microcapsules can be determined using dynamic light scattering and transmission electron microscopy.

In some embodiments or examples, at least 75% by weight of the microcapsules in the composition have a particle size of between about 1 μm to about 100 μm. In an example, at least 75%, at least 85%, or at least 90% by weight of the microcapsules in the composition have a particle size of between about 1 μm to about 100 μm. In another example, at least 75%, at least 85%, or at least 90% by weight of the microcapsules in the composition have a particle size of between about 1 μm to about 50 μm. In another example, at least 75%, at least 85%, or at least 90% by weight of the microcapsules in the composition have a particle size of between about 10 μm to about 50 μm. In another example, at least 75%, at least 85%, or at least 90% by weight of the microcapsules in the composition have a particle size of between about 1 μm to about 30 μm. In another example, at least 75%, at least 85%, or at least 90% by weight of the microcapsules in the composition have a particle size of between about 1 μm to about 5 μm.

In some examples, the compositions are incorporated into various products, including but not limited to pharmaceutical products (i.e. drug delivery), pesticide/insecticide products, nutraceutical products, agrochemicals, and personal products. The composition may also be included in an article, non-limiting examples of which include a dispenser/container. The compositions/articles disclosed herein may be made by combining the microcapsules disclosed herein with the desired adjunct material to form the product. The microcapsules may be combined with the adjuncts material when the microcapsules are in one or more forms, including a slurry form, neat particle form, or spray dried particle form. The microcapsules may be combined with the adjuncts material by methods that include mixing and/or spraying.

### Ionic Shell

The present disclosure provides a microcapsule comprising or consisting of an ionic shell encapsulating an inner fluid core. It will be appreciated that the ionic shell comprises, or is formed from, one or more ionic compounds. For example, the ionic shell may comprise or consist of one or more inorganic calcium phosphate compounds. The microcapsule may comprise or further consist of an inner coating encapsulating the fluid core, wherein the ionic shell encapsulates the inner coating and fluid core. It will be appreciated that the fluid core may be a liquid core or a gel core. The fluid core and/or inner coating can comprise platinum nanoparticles, which can facilitate deposition of the ionic shell on the fluid core or the inner coating of the fluid core.

In another embodiment or example, the ionic shell may further comprise one or more trace elements selected from, but not limited to, titanium, iron, silver, copper, gold, zinc, manganese, strontium, lithium, silicon, fluorine, sodium, barium, and magnesium, forming an ionic shell composite.

The present inventors have unexpectedly found that a continuous substantially impermeable outer ionic shell can be deposited onto the microcapsule by electroless deposition under fast and mild reaction conditions using platinum nanoparticles to catalyse the deposition.

It will be appreciated that the ionic shell comprises, is formed from, or consists of, one or more ionic compounds. It will also be appreciated that ionic compounds are neutral overall, but consist of positively charged “cations” and negatively charged “anions” that can pack together to form a three-dimensional network or crystalline lattice. The ionic compounds may comprise one or more alkaline earth metal. The alkaline earth metal can provide a cation in the ionic compound of the ionic shell. In an example, the alkaline earth metal may be selected from beryllium (Be), magnesium (Mg), calcium (Ca), strontium (Sr), barium (Ba), or combinations thereof. In another example, the ionic shell comprises or consists of one or more ionic compounds selected from phosphates, sulphates, nitrates, silicates, carbonates, or combinations thereof. The ionic compound may be prepared in situ, for example grown or deposited around a fluid core or inner coating of a fluid core, wherein the fluid core can be a liquid core or gel core. In an example, the ionic compound may comprise or consist of an alkaline earth metal in combination with one or more phosphates, sulphates, nitrates, silicates, carbonates, or combinations thereof. In other words, the cation of the ionic compound may be provided by one or more alkaline earth metals and the anion of the ionic compound may be provided by one or more of phosphates, sulphates, nitrates, silicates, and carbonates.

In another example, the ionic compound may be selected from beryllium phosphate, beryllium sulphate, beryllium nitrate, beryllium silicate, beryllium carbonate, magnesium phosphate, magnesium sulphate, magnesium nitrate, magnesium silicate, magnesium carbonate, calcium phosphate, calcium sulphate, calcium nitrate, calcium silicate, calcium carbonate, strontium phosphate, strontium sulphate, strontium nitrate, strontium silicate, strontium carbonate, barium phosphate, barium sulphate, barium nitrate, barium silicate, barium carbonate, or combinations thereof. In another example, the ionic shell may comprise or consist of an inorganic calcium phosphate shell. The ionic shell, or ionic compound thereof, may comprise or consist of a calcium phosphate compound. In another example, the inorganic calcium phosphate shell, or calcium phosphate compound thereof, may be selected from monocalcium phosphate, dicalcium phosphate, tricalcium phosphate, octacalcium phosphate, dicalcium diphosphate, calcium triphosphate, calcium hydroxide phosphate, or combinations thereof.

In some embodiments or examples, the ionic shell may have a thickness of between about 1 nm to about 1000 nm. The thickness of the ionic shell may in a range from about 2 nm to about 900 nm, about 4 nm to about 900 nm, about 6 nm to about 700 nm, about 8 nm to about 600 nm, about 10 nm to about 500 nm, about 12 nm to about 400 nm, about 14 nm to about 300 nm, about 16 nm to about 200 nm, or about 20 nm to about 150 nm. The thickness of the ionic shell may be at least 1 nm, at least 2 nm, at least 4 nm, at least 6 nm, at least 8 nm, at least 10 nm, at least 12 nm, at least 15 nm, at least 20 nm, at least 25 nm, at least 30 nm, at least 35 nm, at least 40 nm, at least 45 nm, or at least 50 nm. The thickness of the ionic shell may be less than 1000 nm, less than 800 nm, less than 600 nm, less than 500 nm, less than 400 nm, less than 300 nm, less than 200, less than 100 nm, or less than 50 nm. The thickness of the ionic shell may be in a range provided by any lower and/or upper limit as previously described.

It will be appreciated that the elemental composition analysis and elemental mapping of the ionic shell may be determined using transmission electron microscopy with energy dispersive X-ray, and the morphology of the ionic shell may be analysed using scanning electron microscopy.

It will be appreciated that the thickness of the ionic shell may have homogeneity of variance. The variance in the thickness of the ionic shell may be in the range of from 4 nm to 150 nm, about 6 nm to about 120 nm, about 8 nm to about 100 nm, or about 10 to about 50 nm. The variance in the thickness of the ionic shell may be at least 0.1 nm, at least 0.5 nm, at least 1.0 nm, at least 5.0 nm, at least 10 nm, at least 15 nm, at least 20 nm, at least 50 nm, at least 100 nm, at least 150 nm, or at least 200 nm. The variance in the thickness of the ionic shell may be less than 300 nm, less than 200 nm, less than 100 nm, less than 80 nm, less than 60 nm, or less than 40 nm. The variance in the thickness of the ionic shell may be in a range provided by any lower and/or upper limit as previously described. In some embodiments or examples, further advantages of the present disclosure may be provided by less variance in the thickness of the ionic shell thickness having been shown with a thicker ionic shell.

The characteristics of the ionic shell may be controlled by adjusting the calcium cation to phosphate anion ratio. In some embodiments or examples the calcium to phosphate ratio may be in the range of about 1:3 to 3:1 or 1:2 to 2:1. In an example, the calcium to phosphate ratio may be about 1:1. In an example, the addition of sodium fluoride to the ionic shell may further facilitate a close-packing of spherical calcium phosphate particles and/or crystals in the form of a single or multiple layers on the surface of the microcapsule.

The inventors have surprisingly found that depositing an ionic shell on a microcapsule, for example depositing an inorganic calcium phosphate shell on a microcapsule, can provide a substantially impermeable microcapsule suitable for a number of applications. In some embodiments or examples, the ionic shell may be substantially impermeable to low molecular weight or volatile “active agent” molecules, for example molecules having a molecular weight of less than about 1000 g·mol⁻¹, 900 g·mol⁻¹, 800 g·mol⁻¹, 700 g·mol⁻¹, 600 g·mol⁻¹, 500 g·mol⁻¹, 400 g·mol⁻¹, 300 g·mol⁻¹, or 200 g·mol⁻¹. In another example, the ionic shell microcapsules can retain low molecular weight active agents present in the liquid core of the microcapsules for up to about 12 hours, 24 hours, 1 day, 2 days, 3 days, 4 days, 5 days, 6 days, 7 days, 2 weeks, 3 weeks, 4 weeks, or 2 months. The impermeability or retention of active agent in the fluid core may be measured by placing the prepared microcapsules into a solution (e.g. chloroform-D) for predetermined time, such as 1 week, and measuring the amount of active agent released into the solution. The retention of active agent within the microcapsule as a weight % of active agent may be at least about 80, 85, 90, 95, 98, 99, 99.5, 99.8, or 99.9.

In some embodiments or examples, the ionic shell may be a densely packed, continuous layer of inorganic material (e.g. calcium phosphate) deposited onto the surface of the microcapsule. The inventors have unexpectedly found that platinum nanoparticles adsorbed or embedded in, on or near the surface of the inner fluid core and/or inner coating can provide an effective catalyst and seed for the deposition of calcium phosphate onto the surface of the microcapsule. In other words, platinum nanoparticles present at the surface of the fluid core and/or inner coating can facilitate formation of the ionic shell. It will be appreciated that if the fluid core comprises an inner coating encapsulating the fluid core, the platinum nanoparticles may be adsorbed or embedded in, on or near the outer surface of the inner coating. It will also be appreciated that the platinum nanoparticles may act as an anchoring point for the ionic shell, i.e. the platinum nanoparticles may provide a site of nucleation for the calcium phosphate material to be deposited as an ionic shell on the surface of the microcapsule. The inventors have unexpectedly found that the ionic shell, such as an inorganic calcium phosphate shell, can auto-catalyse further deposition of the ionic shell over time to form a more continuous or thicker shell around the microcapsule to provide further improved impermeability characteristics.

### Fluid Core

The present inventors have surprisingly found that the microcapsules provide improved impermeability properties and are better able to retain the contents of the inner fluid core without leakage over time.

The microcapsules may comprise an outer ionic shell encapsulating a fluid core. The fluid core may be referred to herein as an “inner fluid core”. In an embodiment, the fluid core may be a liquid core or inner liquid core. The term “liquid core” or “inner liquid core” as used herein refers to a core material formed of one or more components that are liquid at standard ambient temperature and pressure. For example, the liquid core may comprise liquid suspensions, such as a liquid carrier with suspended actives. The term “standard ambient temperature and pressure” refers to a temperature of 25° C. and an absolute pressure of 100 kPa. In another embodiment, the fluid core may be a gel core or inner gel core. The term “gel core” or “inner gel core” as used herein refers to a core material formed of one or more components that are a gel at standard ambient temperature and pressure. For example, the gel core may comprise suspensions, such as a gel carrier with suspended actives.

In some embodiments or examples, the fluid core may comprise between 1% to about 99.9% by weight of the microcapsule. The fluid core (by weight of the microcapsule) may be in the range of from about 5% to about 99.9%, about 10% to about 99.9%, about 20% to about 99.9%, or about 45% to about 99.9%. The inner fluid core (by weight of the microcapsule) may be at least 1%, at least 5%, at least 10%, at least 20%, at least 30%, at least 45%, at least 65%, at least 75%, at least 85%, at least 95%, at least 99%, at least 99.5%, or at least 99.9%. The inner fluid core (by weight of the microcapsule) may be less than about 99.99%, 99.9%, 99.5%, 99%, 98%, 95%, 90%, 85%, 75%, 65%, 45%, 30%, or 20%. The fluid core may be in a range provided by any lower and/or upper limit as previously described.

In some embodiments or examples, the fluid core may comprise one or more active agents. The microcapsules described herein are useful with a wide variety of active agents. In some embodiments or examples, the fluid core may further comprise one or more additives selected from an oil carrier, an aqueous carrier, a water/oil emulsion, solids, and contrast agents. In an embodiment or example, the oil carrier may be selected from oils including, but not limited to, one or more of triglyceride oils, mineral oil, petroleum oil, isopropyl myristate, and silicon oil. It will be appreciated that the oil carrier can be selected from any oil carrier that can dissolve the active ingredient. In an embodiment or example, the aqueous carrier can be water. In an embodiment or example, the solid may be selected from, but not limited to, one or more of polymeric nanoparticles, iron oxide nanoparticles, and silver nanoparticles. In an embodiment or example, the contrast agents can be selected from, but not limited to, one or more of perfluorooctyl bromide, gadolinium-based contrast agents (e.g. gadovist and magnevist), perfluorocarbons, and fluorinated polymers.

In some embodiments or examples, the active agent of the fluid core may be selected from pharmaceuticals, nutraceuticals, pesticides, insecticides, fertilizers, herbicides, perfumes, brighteners, insect repellents, silicones, waxes, flavours, vitamins, fabric softening agents, depilatories, skin care agents, enzymes, probiotics, dye polymer conjugate, perfume delivery system, sensates, attractants, anti-bacterial agents, dyes, pigments, bleaches, flavourants, sweeteners, waxes, UV blockers/absorbers, or combinations thereof. In an embodiment or example, the active agent may be a water soluble active agent or an oil soluble active. In an embodiment or example, the active agent may be a hydrophilic active agent or a hydrophobic active agent. For example, the active agent may have a hydrophilic-lipophilic balance value at any value between 0 and 30.

### Liquid Core

In some embodiments or examples, the liquid core consists of one or more components which are liquid at standard ambient temperature and pressure. In some examples, the liquid core material comprises one or more components which are volatile. Unless otherwise specified, the term “volatile” as used herein refers to those materials that are liquid under ambient conditions and which have a measurable vapour pressure at 25° C. These materials typically have a vapour pressure of greater than about 0.0000001 mm Hg, e.g. from about 0.02 mm Hg to about 20 mm Hg, and an average boiling point typically less than about 250° C.

The liquid core may comprise of a single material or it may be formed of a mixture of different materials. In some embodiments or examples, the liquid core may comprise one or more active agents. The microcapsules described herein are useful with a wide variety of active agents. In some embodiments or examples, the inner liquid core may further comprise one or more additives selected from an oil carrier, an aqueous carrier, solids, a water/oil emulsion, and contrast agents. In an embodiment or example, the oil carrier may be selected from oils including, but not limited to, one or more of triglyceride oils, mineral oil, petroleum oil, isopropyl myristate, and silicon oil. It will be appreciated that the oil carrier can be selected from any oil carrier that can dissolve the active ingredient. In an embodiment or example, the aqueous carrier can be water. In an embodiment or example, the solid may be selected from, but not limited to, one or more of polymeric nanoparticles, iron oxide nanoparticles, and silver nanoparticles. In an embodiment or example, the contrast agents can be selected from, but not limited to, one or more of perfluorooctyl bromide, gadolinium-based contrast agents (e.g. gadovist and magnevist), perfluorocarbons, and fluorinated polymers.

### Gel Core

In some embodiments or examples, the fluid core may be a gel core. The gel core may comprise a gel carrier. In an embodiment or example, the gel core may comprise a gel carrier, one or more active agents, and platinum nanoparticles. In an embodiment or example, the gel carrier may be a crosslinkable polymer. The crosslinkable polymer may be a anionic polymer or a cationic polymer. In an embodiment or example, the anionic polymer may be selected from an alginate, pectin, carboxy methyl cellulose, hyaluronates, or combinations thereof. In another embodiment or example, the cationic polymer may be selected from chitosan, cationic guar, cationic starch, or combinations thereof. In an embodiment, the gel carrier in the gel core may be a hydrogel. It will be appreciated that one or more crosslinkable polymers may form a hydrogel. For example, alginate may form a hydrogel in the presence of divalent cations. The divalent cation may be, for example, calcium, barium, zinc, palladium, platinum, or a combination thereof. For example, the hydrogel may be barium alginate, calcium alginate, or zinc alginate. In one example, the hydrogel may be calcium alginate.

The molecular weight of the gel carrier may be in a range between 32,000 and 400,000 g/mol. For example, the molecular weight may be at least about 30,000, 40,000, 50,000, 60,000, 70,000, 80,000, 90,000, 100,000, 200,000, 300,000 or 400,000. The molecular weight may be in a range of about 30,000 to 400,000, 40,000 to 300,000, 40,000 to 200,000, or 50,000 to 100,000. The molecular weight may be less than about 400,000, 350,000, 300,000, 250,000, 200,000, 100,000, 80,000, 60,000, or 40,000. The number average molecular weight may be in a range provided by any lower and/or upper limit as previously described. It will be appreciated that increasing the molecular weight of gel carrier may improve the physical properties of the gel core. For example, manipulation of the molecular weight and its distribution can independently control the pre-gel solution viscosity and post-gelling stiffness. The elastic modulus of gels can be increased significantly, while the viscosity of the solution minimally raises, by using a combination of high and low molecular weight gel carriers.

In some embodiments or examples, the viscosity of the gel carrier may be in a range between about 20,000 to 200,000 cps. For example, the viscosity may be at least about (cps) 20,000, 50,000, 70,000, 150,000, or 200,000. The viscosity may be less than about (cps) 200,000, 100,000, 80,000, 60,000, 40,000, 30,000, or 20,000. The viscosity may be in a range provided by any lower and/or upper limit as previously described.

The gel carrier may be characterised by a compressive modulus. In some embodiments or examples, the gel core may have a compressive modulus in a range of about 50 to 250 kPa. The compressive modulus may be less than about (kPa) 250, 200, 150, 100, 90, 80, 70, 60, or 50. The compressive modulus may be at least (kPa) 50, 60, 70, 80, 90, 100, 120, 140, 160, 180, 200, 220, or 240. The compressive modulus may be in a range provided by any lower and/or upper limit as previously described.

The gel core may comprise of a single material or it may be formed of a mixture of different materials. In some embodiments or examples, the gel core may comprise a gel carrier. In some embodiments or examples, the gel core may comprise one or more active agents. The microcapsules described herein are useful with a wide variety of active agents. In some embodiments or examples, the inner gel core may further comprise one or more additives selected from an oil carrier, an aqueous carrier, solids, a water/oil emulsion, and contrast agents. In an embodiment or example, the oil carrier may be selected from oils including, but not limited to, one or more of triglyceride oils, mineral oil, petroleum oil, isopropyl myristate, and silicon oil. It will be appreciated that the oil carrier can be selected from any oil carrier that can dissolve the active ingredient. In an embodiment or example, the aqueous carrier can be water. In an embodiment or example, the solid may be selected from, but not limited to, one or more of polymeric nanoparticles, iron oxide nanoparticles, and silver nanoparticles. In an embodiment or example, the contrast agents can be selected from, but not limited to, one or more of perfluorooctyl bromide, gadolinium-based contrast agents (e.g. gadovist and magnevist), perfluorocarbons, and fluorinated polymers.

### Platinum Nanoparticles

The inventors have unexpectedly found that the presence of platinum nanoparticles within an fluid core composition can enable the depositing of a densely packed and/or continuous substantially impermeable ionic shell around a fluid core to form a microcapsule. The deposition may be an electroless deposition under relatively fast and mild reaction conditions. It has been found that the platinum nanoparticles present in the fluid core, or in a polymeric coating encapsulating the fluid core, can effectively catalyse the deposition of an ionic shell to encapsulate the fluid or polymeric coating thereof. It is believed that the platinum nanoparticle catalyst can increase the rate of reaction and act as a seed to localise the deposition of the ionic compound, for example calcium phosphate, as an outer ionic or inorganic shell of the microcapsule.

In some embodiments or examples, the platinum nanoparticles may be present within the inner fluid core prior to deposition of the ionic shell. The platinum nanoparticles may be stabilised platinum nanoparticles, for example platinum nanoparticles coated with one or more polymers. In an embodiment or example, the platinum nanoparticles may be on the surface of the inner coating. For example, the platinum nanoparticle may be at the solid-aqueous interface of the polymer and the external phase.

It will be appreciated that the platinum nanoparticles can comprise or consist of platinum metal (e.g. Pt(0)). In some embodiments or examples, the platinum source for preparing the platinum nanoparticles may be selected from PtCl₂, H₂PtCl₆, K₂PtCl₆, Na₂PtCl₆, K₂PtCl₄, or combinations thereof.

In some embodiments or examples, the ionic shell may be applied by an electroless plating procedure which is catalysed by the platinum nanoparticles.

It will be appreciated that the platinum nanoparticles, or stabilised platinum nanoparticles, will typically have a spheroidal geometry, but they may exist in more complex forms such as rods, stars, ellipsoids, cubes or sheets. In some embodiments or examples, the diameter of the platinum nanoparticle, or stabilised platinum nanoparticle, may be between about 0.1 nm and 200 nm. The diameter of the platinum nanoparticle, or stabilised platinum nanoparticle, may be in the range of from 0.2 nm to 150 nm, about 0.4 nm to about 100 nm, about 0.6 nm to about 50 nm, about 0.8 nm to about 30 nm, about 1.0 nm to about 20 nm, about 2.0 to about 10 nm, 3.0 nm to about 8 nm, or about 4 nm to about 6 nm. The diameter of the platinum nanoparticle, or stabilised platinum nanoparticle, may be at least 0.1 nm, at least 0.2 nm, at least 0.4 nm, at least 0.6 nm, at least 0.8 nm, at least 1.0 nm, at least 2.0 nm, at least 3.0 nm, at least 4.0 nm, at least 5.0 nm, or at least 6.0 nm. The diameter of the platinum nanoparticle, or stabilised platinum nanoparticle, may be less than 300 nm, less than 200 nm, less than 100 nm, less than 80 nm, less than 60 nm, less than 40 nm, less than 20, or less than 10 nm. The diameter of the platinum nanoparticle, or stabilised platinum nanoparticle, may be in a range provided by any lower and/or upper limit as previously described. In some embodiments or examples, further advantages that may be provided by the present disclosure include the use of smaller platinum nanoparticles which may result in the formation of a thinner ionic shell.

### Optional Inner Coating

In some embodiments or examples, the inner fluid core may further comprise an inner coating that encapsulates the fluid core from the ionic shell. The inner coating may be a polymeric shell. In an embodiment, the polymeric shell may comprise or consist of a polymeric material. In an example, the polymeric shell may comprise or consist of a synthetic polymer or a naturally-occurring polymer.

In some embodiments or examples, the synthetic polymer may be selected from nylon, polyethylenes, polyamides, polystyrenes, polyisoprenes, polycarbonates, polyesters, polyureas, polyurethanes, polyolefins, polysaccharides, epoxy resins, vinyl polymers, polyacrylates, or combinations thereof. The polymeric shell may comprise or consist of one or more thermoplastic polymers.

In another embodiment, the naturally-occurring polymer may be selected from silk, wool, gelatin, cellulose, alginate, proteins, or combinations thereof.

In some embodiments or examples, the polymeric shell may comprise or consist of a homopolymer or a copolymer. In an example, the polymeric shell may comprise or consist of a biodegradable polymer.

In some embodiments or examples, the polymeric shell may comprise or consist of a polyester. The polyester may be selected from polyglycolic acid, polylactic acid, polycaprolactone, polyhydroxyalkanoate, polyhydroxybutyrate, polyethylene adipate, polybutylene succinate, polytrimethylcarbonate-co-lactide, or combinations thereof. In an example, the polyester may be a copolymer of polylactic acid and polyglycolic acid. In another example, the polymeric shell may comprise or consist of a polyacrylate. The polyacrylate may be selected from poly(methyl methacrylate) or poly(ethyl methacrylate).

It will be appreciated that the above embodiments and examples of the inner coating comprise or further consist of the platinum nanoparticles as described herein. The platinum nanoparticles can be present in the inner coating to facilitate deposition of the ionic shell around the inner coating to encapsulate the inner coating and fluid core and form a microcapsule according to at least some of the embodiments or examples as described herein.

In some embodiments or examples, the inner coating has a thickness of between about 10 nm to about 5000 nm. The thickness of the inner coating may be in the range of from about 12 nm to 4500 nm, about 14 nm to about 4000 nm, about 16 nm to about 3500 nm, about 18 nm to about 3000 nm, about 20 nm to about 2500 nm, about 25 nm to about 2000 nm, or about 30 nm to about 1500 nm. The thickness of the inner coating may be at least 10 nm, at least 20 nm, at least 40 nm, at least 60 nm, at least 80 nm, at least 100 nm, at least 200 nm, at least 400 nm, at least 800 nm, at least 1000 nm, or at least 1500 nm. The thickness of the inner coating may be less than 2000 nm, less than 1800 nm, less than 1500 nm, less than 1200 nm, less than 1000 nm, less than 800 nm, less than 500 nm, less than 200 nm, less than 100 nm, less than 80 nm, less than 50 nm, or less than 40 nm. The thickness of the inner coating may be in a range provided by any lower and/or upper limit as previously described.

In some embodiments or examples, the inner coating has a thickness measured by ratio of radius of fluid core to inner coating. For example, the ratio of the radius of the inner coating to the fluid core may be about 0.01 to about 0.3 (e.g. 1:100 to 3:10). The ratio may be in the range of about 0.015 to 0.28, about 0.018 to 0.25, about 0.02 to 0.22, about 0.023 to 0.2, about 0.025 to 0.18, about 0.03 to 0.15. The ratio may be at least 0.013, at least 0.015, at least 0.018, at least 0.02, at least 0.023, at least 0.025, at least 0.03, at least 0.05, at least 0.08, at least 0.1, or at least 0.15. The ratio may be less than 0.3, less than 0.28, less than 0.25, less than 0.22, less than 0.2, less than 0.18, less than 0.15, less than 0.1, less than 0.08, less than 0.05, or less than 0.03. The ratio may be in a range provided by any lower and/or upper limit as previously described.

In some embodiments or examples, the fluid core comprises about 50% to about 95% by weight of the total inner fluid core and inner coating. The fluid core (by weight of the total inner fluid core and inner coating) may be in the range of about 60% to 94%, about 70% to 93%, about 75% to 92%, or about 80% to 90%. The fluid core (by weight of the total inner fluid core and inner coating) may be at least 50%, at least 55%, at least 60%, at least 65%, at least 70%, at least 75%, at least 80%, or at least 85%. The fluid core (by weight of the total inner fluid core and inner coating) may be less than 95%, less than 90%, less than 85%, less than 80%, less than 75%, or less than 70%. The fluid core may be in a range provided by any lower and/or upper limit as previously described.

In some embodiments or examples, the ratio by weight of the fluid core to inner coating may be between about 10:1 to about 0.05:1. The ratio by weight of the fluid core to inner coating may be in the range of from about 9:1 to about 0.1:1, about 8:1 to about 0.2:1, about 7:1 to about 0.5:1, or about 6:1 to about 1:1. The ratio by weight of the fluid core to inner coating may be at least 0.05:1, at least 0.1:1, at least 0.2:1, at least 0.5:1, at least 1:1, at least 2:1, or at least 4:1. The ratio by weight of the fluid core to inner coating may be less than 8:1, less than 6:1, less than 4:1, less than 2:1, less than 1:1, less than 0.5:1, less than 0.2:1, or less than 0.1:1. The ratio by weight of the fluid core to inner coating may be in a range provided by any lower and/or upper limit as previously described.

### Process for Preparing Microcapsules

The microcapsules defined by the present disclosure may be formed by emulsifying the inner fluid core materials into droplets followed by encapsulating the inner fluid core with an ionic shell. In some embodiments or examples, the ionic shell may be deposited as a densely packed and/or continuous layer over the fluid core. In an example, the ionic shell may be deposited as a densely packed and/or continuous layer over the inner fluid core by electroless plating catalysed by platinum nanoparticles present at the inner fluid core interface.

In some embodiments or examples, the process comprises incorporating or embedding the platinum nanoparticles within the inner fluid core prior to deposition of the outer inorganic shell.

In other embodiments or examples, the process further comprises forming an inner coating around the droplets, prior to formation of the ionic shell around the inner coating. In an embodiment, the process comprises encapsulating the fluid core by an inner coating using an emulsification process prior to deposition of the ionic shell on to the inner coating. In an example, the platinum nanoparticles present in the fluid core composition are adsorbed within the inner coating to form a discontinuous layer platinum nanoparticles on the surface of the inner coating during the emulsification process. The ionic shell may then be deposited on to the inner coating to further encapsulate the fluid core and form a microcapsule according to at least some embodiments or examples as described herein.

### Microcapsule Synthesis

In some embodiments or examples, the microcapsules may be formed by emulsifying the fluid core into droplets. In other embodiments or examples, the microcapsules may be formed by emulsifying the inner fluid core into droplets and forming an inner coating around the droplets. It will be appreciated that microencapsulation of the inner fluid core may be provided using a variety of methods known in the art, including, for example, coacervation methods, in situ polymerisation methods or interfacial polymerisation methods.

In some embodiments or examples, the microcapsules may be prepared by a coacervation method which involves oil-in-water emulsification followed by solvent extraction. Such procedures are known in the art (see, e.g., Loxley et al., Journal of Colloid and Interface Science, vol. 208, pp. 49-62, 1998) and involve the use of a non-aqueous phase comprising a polymeric material that is capable of forming an inner coating, a poor solvent for the polymeric material, and a co-solvent which is a good solvent for the polymeric material. The non-aqueous and aqueous phases are emulsified, forming an oil-in-water emulsion comprising droplets of the non-aqueous phase dispersed in the continuous aqueous phase. In some embodiments, the co-solvent may then be partially or wholly extracted from the non-aqueous phase, causing the polymeric material to precipitate around the poor solvent, thereby encapsulating the poor solvent.

In some embodiments or examples, the microcapsules may be prepared by: (i) providing a non-aqueous phase comprising a fluid core, and a co-solvent; (ii) providing an aqueous phase; (iii) emulsifying the non-aqueous phase and the aqueous phase to form an emulsion comprising droplets of the non-aqueous phase dispersed within the aqueous phase; and (iv) extracting at least a portion of the co-solvent from the non-aqueous phase such that droplets comprising the fluid core material are formed, thereby encapsulating the fluid core material.

In some embodiments or examples, the microcapsules may be prepared by: (i) providing a non-aqueous phase comprising a polymeric material that is capable of forming an optional inner coating, a fluid core which is a poor solvent for the polymeric material, and a co-solvent which is a good solvent for the polymeric material; (ii) providing an aqueous phase; (iii) emulsifying the non-aqueous phase and the aqueous phase to form an emulsion comprising droplets of the non-aqueous phase dispersed within the aqueous phase; and (iv) extracting at least a portion of the co-solvent from the non-aqueous phase such that the polymeric material precipitates around droplets comprising the fluid core material, thereby encapsulating the fluid core material.

In another embodiment or example, the microcapsules may be formed by forming a gel core. The gel core may comprise a gel carrier wherein the gel carrier may comprise one or more active agents and platinum nanoparticles. In an embodiment or example, the gel carrier in the gel core may be a crosslinkable polymer, as described herein. In an embodiment, the gel carrier in the gel core may be a hydrogel. It will be appreciated that one or more crosslinkable polymers may form a hydrogel, i.e. a hydrogel may be composed of three-dimensional networks of hydrophilic polymers with a water content of about 40 to 90 wt. %. In some embodiments, the hydrogel may comprise at least about 0.5, 1, 5, 10, 20, 30, 40, 50, 60, 70, 80, 90, 95 or 99 wt. % water. In some embodiments, the hydrogel may comprise less than about 99, 90, 80, 70, 60, 50, 40, 30, 20, 10, 5, 1, or 0.5 wt. % water. Combinations of these wt. % values to form various ranges are also possible, for example the hydrogel may comprise between about 40 wt. % to about 99 wt. % water. For example, alginate may form a hydrogel in the presence of divalent cations. The divalent cation may be, for example, calcium, barium, zinc, palladium, platinum, or a combination thereof. For example, the hydrogel may be barium alginate, calcium alginate, or zinc alginate. In one example, the hydrogel may be calcium alginate.

It will be appreciated that chemical and/or physical cross-linking of hydrophilic polymers are typical approaches to forming hydrogels, and their physicochemical properties are highly dependent on the cross-linking type and cross-linking density, in addition to the molecular weight and chemical composition of the polymers. In some embodiments or examples, the gel core may be prepared from an aqueous alginate solution combined with a solution comprising a ionic cross-linking agent, e.g. divalent cations (i.e., Ca²⁺, Ba²⁺, Zn²⁺). It will be appreciated that the divalent cations may bind solely to the guluronate residues of the alginate chains, as the structure of the guluronate residues may allow a high degree of coordination of the divalent ions. The guluronate residues of one polymer may then form junctions with the guluronate residues of adjacent polymer chains resulting in a gel structure, i.e. forming a gel core. For example, the source of divalent cations may be selected from the group comprising calcium chloride, calcium sulfate, calcium carbonate, barium chloride, barium sulfate, barium carbonate, zinc chloride, zinc sulfate, zinc carbonate, palladium chloride, palladium sulfate, palladium carbonate, platinum chloride, platinum sulfate, platinum carbonate. In one example, the source of divalent cations may be calcium chloride.

It will be appreciated that the inner coating, polymeric material and inner fluid core, as described in the above processes may be provided by any of the embodiments or examples as described herein for the inner coating, polymeric material and fluid core.

In some embodiments or examples, the co-solvent is a volatile material. In an embodiment, the co-solvent may be dichloromethane, and is extracted from the non-aqueous phase by evaporation. In this case, precipitation of the polymeric material may be aided by heating the emulsion to promote evaporation of the co-solvent. For instance, the method may be carried out at a temperature of at least 30° C.

In some embodiments or examples, at least one of the aqueous and non-aqueous phases comprises an emulsifier. In an embodiment, the aqueous phase comprises an emulsifier. It will be appreciated that the emulsifier may comprise any of the embodiments or examples described herein for the emulsifier. In an example, the emulsifier may be selected from poly(vinyl alcohol) (PVA), poly(vinyl pyrrolidone) (PVP), cetyl trimethylammonium bromide (CTAB) or combinations thereof.

In some embodiments or examples, the inner coating is a polymeric shell. The polymeric shell can be formed by an interfacial polymerisation process. In an embodiment, the polymeric shell may be prepared by an interfacial polymerisation process which involves the use of a non-aqueous phase comprising the inner fluid core and one or more oil-soluble monomers; and an aqueous phase comprising one or more water-soluble monomers and an emulsifier. In an example, the non-aqueous and aqueous phases are emulsified to form an emulsion comprising droplets of the non-aqueous phase dispersed within the aqueous phase. The monomers are then polymerised, typically by heating, with polymerisation occurring at the interface between the non-aqueous phase and the aqueous phase.

In an alternate embodiment, the polymeric shell may be provided by interfacial polymerisation of a pre-polymer. Such processes may be used to prepare a range of different polymeric shell materials. For example, a polymeric shell comprising a copolymer of polylactic acid and polyglycolic acid may be prepared by such a process.

In some embodiments or examples, the interfacial polymerisation process may include the presence of a free radical initiator. In an embodiment, the free radical initiators may include azo initiators, peroxide, alkyl peroxides, dialkyl peroxides, peroxyesters, peroxycarbonates, peroxyketones and peroxydicarbonates. In some examples, the free radical initiator may be selected from 2,2′-azobis-(2,4-dimethylpentanenitrile), 2,2′-azobis-(2-methyl-butyronitrile), and mixtures thereof. It will be appreciated that the free radical initiator may be present in the aqueous phase, the non-aqueous phase, or both.

In some embodiments or examples, the microcapsules may be prepared by an in situ polymerisation process. Such processes are known in the art and generally involve preparing an emulsion comprising droplets of the fluid core material dispersed in a continuous phase comprising a precursor material which can be polymerised to form a polymeric shell; and polymerising the precursor material to form a polymeric shell, thereby encapsulating the liquid droplets. The polymerisation process is similar to that of interfacial polymerisation processes, except in that no precursor materials for the polymeric shell are included in the fluid core material for the in situ polymerisation processes. Thus, polymerisation occurs solely in the continuous phase, rather than on either side of the interface between the continuous phase and the inner fluid core.

In some embodiments or examples, the precursor material for the polymeric shell may be selected from pre-polymer resins such as urea resins, melamine resins, acrylate esters, and isocyanate resins. In an example, the polymeric shell may be formed by the polymerisation of a precursor material selected from melamine-formaldehyde resins; urea-formaldehyde resins; monomeric or low molecular weight polymers of methylol melamine; monomeric or low molecular weight polymers of dimethylol urea or methylated dimethylol urea; and partially methylated methylol melamine.

In an example, melamine-formaldehyde resins or urea-formaldehyde resins may be used as the precursor material. Procedures for preparing microcapsules comprising such precursor materials are known in the art (see, e.g., U.S. Pat. Nos. 3,516,941, 5,066,419 and 5,154,842). The capsules are made by first emulsifying the inner fluid core as small droplets in an aqueous phase comprising the melamine-formaldehyde or urea-formaldehyde resin, and then allowing the polymerisation reaction to proceed along with precipitation at the oil-water interface.

It will be appreciated that in each of the emulsification processes described herein, emulsification can be conducted using any suitable mixing device known in the art. For example, a homogeniser, colloid mill, ultrasonic dispersion device, or ultrasonic emulsifier may be used. In an example, a homogeniser is used.

Platinum Nanoparticle Synthesis and Embedding into the Microcapsule

In some embodiments or examples, the platinum nanoparticles are adsorbed within and/or onto the inner fluid core or alternatively adsorbed within and/or onto the inner coating in the form of a discontinuous layer, prior to application of the outer ionic shell. It will be appreciated that the term “discontinuous layer” means that the surface of the inner fluid core or surface of the inner coating comprises regions comprising adsorbed platinum nanoparticles and regions in which adsorbed platinum nanoparticles are absent. The platinum nanoparticles may be distributed over the surface of the fluid core or inner coating in a substantially uniform manner.

It will be appreciated that the deposition of the platinum nanoparticles may occur in various ways including, but not limited to, adsorption of charge-stabilised platinum nanoparticles, adsorption of sterically-stabilised platinum nanoparticles, or deposition by reduction in situ.

In some embodiments or examples, the platinum nanoparticles are charge-stabilised nanoparticles which are adsorbed on the inner fluid core or the inner coating encapsulating the inner fluid core. It will be appreciated that the charge-stabilised platinum nanoparticles comprise a charged species adsorbed on the surface thereof. Since the stabiliser is a charged species, it will impart a charged surface to the nanoparticles which can be exploited in order to adsorb the platinum nanoparticles to the surface of the inner fluid core or inner coating encapsulating the inner fluid core. In some examples, the platinum nanoparticles are adsorbed on the inner fluid core or inner coating encapsulating the inner fluid core by electrostatic interaction.

In some embodiments or examples, the platinum nanoparticles may be adsorbed on a surface-modifying agent that forms part of the inner fluid core or inner coating encapsulating the inner fluid core. In an embodiment, the surface-modifying agent may be adsorbed on and/or absorbed within the inner fluid core or inner coating encapsulating the inner liquid core. In an example, the inner liquid core or the inner coating encapsulating the inner fluid core may be formed by an emulsification process in which the surface-modifying agent was employed as an emulsifier, with the emulsifier being retained in the inner fluid core or inner coating encapsulating the inner fluid core.

In some embodiments or examples, the surface-modifying agent may provide a charged surface which may be used to electrostatically attract and adsorb the charge-stabilised platinum nanoparticles on to the inner fluid core or inner coating encapsulating the inner fluid core.

In other embodiments or examples, the platinum nanoparticles may be charge-stabilised by an anionic stabiliser. In an embodiment, the anionic stabiliser may be selected from borohydride anions and citrate anions. In another embodiment, the anionic stabiliser may be an anionic surfactant. For example, the anionic surfactant may be selected from sodium dodecyl sulfate, sodium laureth sulfate, dodecyl benzene sulfonic acid, perfluorooctanesulfonate, dioctyl sodium sulfosuccinate and sodium stearate. In an example, the charge-stabilised platinum nanoparticles are borohydride-stabilised or citrate-stabilised platinum nanoparticles.

In some embodiments or examples, the platinum nanoparticles may be stabilised by an anionic stabiliser and the inner fluid core or inner coating encapsulating the inner fluid core comprises a non-ionic surface-modifying agent. In an embodiment, the surface-modifying agent may be a non-ionic polymer. For example, the non-ionic polymer may be selected from poly(vinyl alcohol) and poly(vinyl pyrrolidone). In an example, the non-ionic polymer may be poly(vinyl pyrrolidone).

In some embodiments or examples, the platinum nanoparticles may be stabilised by an anionic stabiliser and the inner fluid core or inner coating encapsulating the inner fluid core comprises a cationic surface-modifying agent. In an embodiment, the surface-modifying agent may be a cationic surfactant or a cationic polymer. In an example, the cationic surfactant may be selected from alkyl ammonium surfactants such as cetyl trimethylammonium bromide, dodecyl dimethylammonium bromide, cetyl trimethylammonium chloride, benzalkonium chloride, cetylpyridinium chloride, dioctadecyl dimethylammonium chloride and dioctadecyl dimethylammonium bromide. In another example, the cationic polymer may be selected from poly(diethylaminoethyl methacrylate), poly(dimethylaminoethyl methacrylate), poly(tertiarybutylaminoethyl methacrylate) and di-block copolymers formed of a first block comprising a poly(aminoalkyl acrylate) and a second block comprising a poly(alkyl acrylate). For example, the surface-modifying agent may be cetyl trimethylammonium bromide.

In some embodiments or examples, the platinum nanoparticle may be charge-stabilised by a cationic stabiliser. In an embodiment, the cationic stabilisers may be selected from cationic surfactants such as quaternary ammonium surfactants. In an example, the quaternary ammonium surfactants may be cetyl trimethylammonium bromide, tetraoctylammonium bromide or dodecyl trimethylammonium bromide. In another example, the quaternary ammonium surfactants be esterquats, for example, quaternary ammonium surfactants containing an ester group.

It will be appreciated that if the platinum nanoparticles are stabilised by a cationic stabiliser, the surface of the inner fluid core or inner coating encapsulating the inner fluid core may be neutral or anionic. In some embodiments or examples, the inner fluid core or inner coating encapsulating the inner fluid core may have a substantially neutral surface having a zeta potential of from −10 mV to +10 mV. In a particular example, the zeta potential may be in a range from −5 mV to +5 mV. In other embodiments or examples, the inner fluid core or inner coating encapsulating the inner fluid core may have a positively charged surface. For example, the zeta potential may be in a range from −20 mV to −150 mV. In a particular example, the zeta potential may be in a range from −30 mV to −90 mV.

In some embodiments or examples, the platinum nanoparticles may be stabilised by a cationic stabiliser and the inner fluid core or inner coating encapsulating the inner fluid core may comprise or consist of a non-ionic surface-modifying agent. In an embodiment, the surface-modifying agent may a non-ionic polymer. In an example, the non-ionic polymer may be selected from poly(vinyl alcohol) and poly(vinylpyrrolidone). In an example, the non-ionic polymer may be poly(vinylpyrrolidone).

In some embodiments or examples, the platinum nanoparticles may be stabilised by a cationic stabiliser and the inner fluid core or inner coating encapsulating the inner fluid core may comprise or consist of an anionic surface-modifying agent. In an embodiment, the surface-modifying agent may be an anionic surfactant or an anionic polymer. In an example, the anionic surfactants may be selected from sodium dodecyl sulfate, sodium laureth sulfate, dodecyl benzene sulfonic acid, dioctyl sodium sulfosuccinate, perfluorooctanesulfonate, dioctyl sodium sulfosuccinate or sodium stearate. In an example, the anionic polymers may be selected from polyacids such as poly(acrylic acid) and poly(methacrylic acid).

In some embodiments or example, the platinum nanoparticles may be charge-stabilised by a zwitterionic stabiliser. In some embodiments, the zwitterionic stabiliser may be a zwitterionic surfactant. In an example, the zwitterionic surfactants may be aminobetaines, imidazoline derivatives and phospholipids.

It will be appreciated that the charge-stabilised platinum nanoparticles may be prepared using suitable procedures known in the art (see, e.g., G. Frens, Nature, 1973, 241, 20-22). Such procedures will typically involve reducing metal ions in solution in the presence of charged stabiliser. Thus, the charge-stabilised nanoparticles may be obtained by providing a solution comprising ions of platinum source and a charged stabiliser, and reducing the ions to form platinum nanoparticles which are charge-stabilised by the stabiliser.

In some embodiments or examples, the platinum ions in solution are reduced by a reducing agent which becomes the charged stabiliser e.g. by sodium borohydride or by sodium citrate. For example, borohydride-stabilised platinum nanoparticles may be prepared by contacting an aqueous solution of hexachloroplatinic acid with sodium borohydride.

The resulting charge-stabilised platinum nanoparticles may then be contacted with the inner fluid core or inner coating encapsulating the inner fluid core under appropriate conditions, e.g. at ambient temperature. The microcapsules may then be washed to remove any unbound particles.

In some embodiments or examples, the platinum nanoparticles may be deposited by adsorbing sterically-stabilised platinum nanoparticles of platinum onto the surface of the inner fluid core or the inner coating encapsulating the inner fluid core. In an embodiment, the sterically-stabilised platinum nanoparticles may comprise or consist of a polymer or other macromolecule which is adsorbed on the surface of the platinum nanoparticle, forming a protective sheath around the particles and minimising aggregation. The size of the steric stabiliser can be exploited in order to adsorb the platinum nanoparticles onto the surface of the inner fluid core or inner coating encapsulating the inner fluid core. In an example, the platinum nanoparticles are adsorbed on the inner fluid core or inner coating encapsulating the inner fluid core by steric interaction.

In some embodiments or examples, the platinum nanoparticles may be sterically-stabilised by a polymeric stabiliser. In an embodiment, the polymer may comprise or consist of one or more groups selected from carboxyl, hydroxyl, amine, and ester groups. The polymer may be a homopolymer or a copolymer (e.g. a graft copolymer or a block copolymer). For example, suitable polymers may be selected from poly(ethylene oxide), polyethylene glycol, poly(acrylic acid), poly(acrylamide), poly(ethylene imine), poly(vinyl alcohol), carboxymethyl cellulose, chitosan, guar gum, gelatin, amylose, amylopectin, and sodium alginate.

In some embodiments or examples, the polymeric stabiliser may have a weight average molecular weight in the range of from about 5 kDa to about 100 kDa. The polymeric stabiliser may have a weight average molecular weight in the range of from about 5 kDa to about 100 kDa, about 10 kDa to about 80 kDa, about 15 kDa to about 60 kDa, or about 20 kDa to about 40 kDa. The polymeric stabiliser may have a weight average molecular weight at least 5 kDa, at least 10 kDa, at least 15 kDa, at least 20 kDa, or at least 30 kDa. The polymeric stabiliser may have a weight average molecular weight less than 100 kDa, less than 80 kDa, less than 60 kDa, or less than 40 kDa. The polymeric stabiliser may have a weight average molecular weight in a range provided by any lower and/or upper limit as previously described.

In some embodiments or examples, the polymeric stabiliser may be a non-ionic polymer. In an embodiment, the non-ionic polymer may be selected from poly(vinyl alcohol), poly(vinyl propylene), poly(ethylene glycol) or poly(vinyl pyrrolidone). In an example, the non-ionic polymer may be poly(vinyl pyrrolidone). For example, the polymeric stabiliser may be poly(vinyl pyrrolidone).

In some embodiments or examples, the polymeric stabiliser may be a cationic polymer. In an embodiment, the cationic polymer may be selected from poly(allyl amine) polymers. For example, the cationic polymer may be poly(allyl amine hydrochloride).

In some embodiments or examples, the polymeric stabiliser may be an anionic polymer. In an embodiment, the anionic polymer may be selected from polyacids. In an example, the anionic polymer may be poly(acrylic acid) or poly(methacrylic acid).

In some embodiments or examples, the nanoparticles may be sterically-stabilised by a polymeric surfactant. In an embodiment, the polymeric surfactant may be selected from polyoxyalkylene glycol alkyl ethers (e.g. polyoxyethylene glycol alkyl ethers and polyoxypropylene glycol alkyl ethers), sorbitan esters (e.g. polysorbates), fatty acid esters, poly(isobutenyl) succinic anhydride amine derivatives or amine oxides.

In some embodiments or examples, the polymeric surfactant may have a weight average molecular weight in the range of from about 5 kDa to about 100 kDa. The polymeric surfactant may have a weight average molecular weight in the range of from about 5 kDa to about 100 kDa, about 10 kDa to about 80 kDa, about 15 kDa to about 60 kDa, or about 20 kDa to about 40 kDa. The polymeric surfactant may have a weight average molecular weight at least 5 kDa, at least 10 kDa, at least 15 kDa, at least 20 kDa, or at least 30 kDa. The polymeric surfactant may have a weight average molecular weight less than 100 kDa, less than 80 kDa, less than 60 kDa, or less than 40 kDa. The polymeric surfactant may have a weight average molecular weight in a range provided by any lower and/or upper limit as previously described.

In some embodiments or examples, the platinum nanoparticles may be adsorbed on a surface-modifying agent that forms part of the inner fluid core or inner coating encapsulating the inner fluid core. In an embodiment, the surface-modifying agent may be adsorbed on and/or absorbed within the inner fluid core or inner coating encapsulating the inner fluid core. In an example, the inner fluid coating or the inner coating was obtained by an emulsification process in which the surface-modifying agent was employed as an emulsifier, with the emulsifier being retained in the inner fluid core or inner coating encapsulating the inner fluid core. In an example, the sterically-stabilised platinum nanoparticles may bind via steric interactions to the surface-modifying agent.

In some embodiments or examples, the surface-modifying agent may be a non-ionic surface-modifying agent. For example, a non-ionic surfactant or a non-ionic polymer. In an embodiment, the non-ionic polymer may be poly(vinyl alcohol) or poly(vinyl pyrrolidone). In an example, the non-ionic polymer may be poly(vinyl alcohol).

In some embodiments or examples, the surface-modifying agent may be a cationic surface-modifying agent. For example, a cationic surfactant or a cationic polymer. In an embodiment, the cationic surfactant may be selected from cetyl trimethylammonium bromide, dodecyl dimethylammonium bromide, cetyl trimethylammonium chloride, benzalkonium chloride, cetylpyridinium chloride, dioctadecyl dimethylammonium chloride or dioctadecyl dimethylammonium bromide. In an example, the cationic surface-modifying agent may be cetyl trimethylammonium bromide.

In some embodiments or examples, the surface-modifying agent may be an anionic surface-modifying agent. For example, an anionic surfactant or an anionic polymer. In an embodiment, the anionic surfactant may be selected from sodium dodecyl sulfate, sodium laureth sulfate, dodecyl benzene sulfonic acid, dioctyl sodium sulfosuccinate, perfluorooctanesulfonate, dioctyl sodium sulfosuccinate or sodium stearate. In an example, the anionic polymer may be a polyacid such as poly(acrylic acid) or poly(methacrylic acid).

It will be appreciated that suitable procedures for preparing the sterically-stabilised platinum nanoparticles are known in the art (see, e.g., Horiuchi et al., Surface and Coatings Technology, 2010, 204, 3811-3817). For example, sterically-stabilised platinum nanoparticles may be prepared by reducing platinum ions in solution in the presence of a stabiliser.

In some embodiments or examples, the platinum nanoparticles may be adsorbed on to the inner fluid core or inner coating by contacting the inner fluid core or inner coating encapsulating the inner fluid core with a slurry comprising said particles. In an embodiment, the platinum nanoparticles are present in the slurry in an amount of more than 0.2% by weight and the slurry comprises less than 0.01% by weight of unbound stabiliser.

In some embodiments or examples, the contacting may take place under ambient conditions. However, in order to facilitate adsorption of the platinum nanoparticles onto the inner fluid core or inner coating encapsulating the inner fluid core, the inner fluid core or inner coating encapsulating the inner fluid core may be heated so as to enhance penetration of the sterically-stabilised platinum nanoparticles within the inner fluid core or inner coating encapsulating the inner fluid core. The temperature may be provided close to or below the glass transition temperature of the polymer of the inner coating and/or fluid core, for example about 80° C. for PMMA or about 40° C. for PLGA. In some embodiments, the inner fluid core or inner coating encapsulating the inner fluid core may be heated to a temperature of from 10° C. to 100° C. The temperature may be in the range of from about 15° C. to about 95° C., about 20° C. to about 90° C., about 25° C. to about 85° C., about 30° C. to about 80° C., about 35° C. to about 75° C., about 40° C. to about 70° C., or about 45° C. to about 65° C. The temperature may be at least 15° C., at least 20° C., at least 25° C., at least 30° C., at least 35° C., at least 40° C., at least 45° C., at least 50° C., at least 55° C., at least 60° C., at least 65° C., at least 70° C., at least 75° C., or at least 80° C. The temperature may be less than 90° C., less than 85° C., less than 80° C., less than 75° C., less than 70° C., less than 75° C., less than 70° C., less than 65° C., less than 60° C., less than 55° C., less than 50° C., less than 45° C., or less than 40° C. The temperature may be in a range provided by any lower and/or upper limit as previously described.

In some embodiments or examples, the platinum nanoparticles may be adsorbed on to the inner fluid core or inner coating by contacting the inner fluid core or inner coating encapsulating the inner fluid core with a solution comprising platinum ions and a reducing agent. It will be appreciated that the presence of the reducing agent causes the platinum ions to be reduced in situ. As the platinum ions are reduced, they precipitate from the solution as metal particles and seek to lower the energy of the system by adsorbing onto the inner fluid core or inner coating encapsulating the inner fluid core. It will be appreciated that platinum may also be adsorbed onto the inner fluid core or inner coating encapsulating the inner fluid core during the deposition process in the form of ions which have not been reduced by the reducing agent.

In an embodiment, the reducing agent that is contacted with the inner fluid core or inner coating encapsulating the inner fluid core may be in solution. For example, the reducing agent may be added to a solution comprising the platinum ions and the inner fluid core or inner coating encapsulating the inner fluid core. Thus, deposition of the platinum nanoparticles on the surface of the inner fluid core or inner coating encapsulating the inner fluid core may be achieved by preparing an aqueous solution comprising platinum ions and inner fluid core or inner coating encapsulating the inner fluid core. A reducing agent is then added to the solution, resulting in reduction of the platinum ions and the precipitation of platinum nanoparticles onto the surface of the inner fluid core or inner coating encapsulating the inner fluid core. The reaction is allowed to progress for a time sufficient to allow the desired deposition of the platinum nanoparticles on the surface of the inner fluid core or inner coating encapsulating the inner fluid core. The microcapsules may then be washed, separated from the other reagents and redispersed in water. The deposition process may be carried out at room temperature.

In some embodiments or examples, the platinum nanoparticle may be adsorbed on a surface-modifying agent that is present in the inner fluid core or inner coating encapsulating the inner fluid core. In an embodiment, the surface-modifying agent may be adsorbed on and/or absorbed within the inner fluid core or inner coating encapsulating the inner fluid core. In an example, the inner fluid core or inner coating encapsulating the inner fluid core was obtained by an emulsification process in which the surface-modifying agent was employed as an emulsifier, with the emulsifier being retained in the inner fluid core or inner coating encapsulating the inner fluid core. In an embodiment, the platinum nanoparticles may be adsorbed to the inner fluid core or inner coating encapsulating the inner fluid core by one or more interactions selected from steric interactions and electrostatic interactions.

In some embodiments or examples, the surface-modifying agent may be a non-ionic surface-modifying agent. For example, the non-ionic surface-modifying agent may be a non-ionic polymer. In an embodiment, the non-ionic polymer may be selected from poly(vinyl alcohol) or poly(vinyl pyrrolidone). In an example, the non-ionic polymer may be poly(vinyl alcohol).

In some embodiments or examples, the surface-modifying agent may be a cationic surface-modifying agent. For example, a cationic surfactant or a cationic polymer. In an embodiment, the cationic surfactant may be selected from cetyl trimethylammonium bromide, dodecyl dimethylammonium bromide, cetyl trimethylammonium chloride, benzalkonium chloride, cetylpyridinium chloride, dioctadecyl dimethylammonium chloride or dioctadecyl dimethylammonium bromide. In an example, the cationic surface-modifying agent may be cetyl trimethylammonium bromide.

In some embodiments or examples, the surface-modifying agent may be an anionic surface-modifying agent. For example, an anionic surfactant or an anionic polymer. In an embodiment, the anionic surfactant may be selected from sodium dodecyl sulfate, sodium laureth sulfate, dodecyl benzene sulfonic acid, dioctyl sodium sulfosuccinate, perfluorooctanesulfonate, dioctyl sodium sulfosuccinate or sodium stearate. In an example, the anionic polymer may be a polyacid such as poly(acrylic acid) or poly(methacrylic acid).

### Ionic Shell Deposition

In some embodiments or examples, the ionic shell is deposited by an electroless plating procedure catalysed by the platinum nanoparticles described herein. In an embodiment, platinum nanoparticles catalyse an electroless plating process.

Further advantages can be provided when platinum nanoparticles are embedded within the fluid core or inner coating, which can provide an effective catalyst and seed for the deposition of an ionic compound such as calcium phosphate onto the surface of the microcapsule. The calcium phosphate may be prepared, for example, in-situ and formed from, or consists of, one or more ionic compounds. The ionic compound may be prepared in situ, for example grown or deposited around the fluid core or inner coating of a fluid core. It will be appreciated that the fluid core may be a liquid core or a gel core.

In some embodiments or examples, following the adsorption of the platinum nanoparticles on the surface of the fluid core or the surface of the inner coating encapsulating the fluid core, a film of the ionic shell may be formed on the discontinuous layer of platinum nanoparticle, thereby coating the surface of the fluid core or the surface of the inner coating encapsulating the fluid core with a densely packed and/or continuous inorganic coating that surrounds the microcapsule. In an embodiment or example, the ionic shell may be calcium phosphate that has been prepared in-situ.

In some embodiments or examples, following the adsorption of the platinum nanoparticles on the surface of the fluid core or the surface of the inner coating encapsulating the fluid core, a film of the ionic shell composite may be formed on the discontinuous layer of platinum nanoparticle, thereby coating the surface of the fluid core or the surface of the inner coating encapsulating the fluid core with a densely packed and/or continuous inorganic coating comprising one or more trace elements selected from, but not limited to, titanium, iron, silver, copper, gold, zinc, manganese, strontium, lithium, silicon, fluorine, sodium, barium, or magnesium, that surrounds the microcapsule. In an embodiment or example, the ionic shell composite may be calcium phosphate comprising one or more trace elements selected from, but not limited to, titanium, iron, silver, copper, gold, zinc, manganese, strontium, lithium, silicon, fluorine, sodium, barium, or magnesium, that has been prepared in-situ.

It will be appreciated that the composition or properties of the ionic shell, such as thickness of the ionic shell, may be provided by any one or more of the embodiments or examples as previously described herein for the ionic shell.

The ionic shell may be formed by an electroless plating process in which the deposition of an ionic compound (e.g. calcium phosphate) may be catalysed by the adsorbed platinum nanoparticles. In an embodiment, the electroless deposition process may comprise contacting the microcapsules onto which the platinum nanoparticle have been deposited with a solution of calcium ions in the presence of a reducing agent (phosphate ions), in the absence of an electric current. In an embodiment, the reducing agent may be the phosphate source and the electroless plating may be performed under acidic or alkaline conditions. In an embodiment, the electroless plating may be performed under acidic conditions. In an example, the acid may be selected from succinic acid. In another embodiment, the electroless plating may be performed using thiourea. In another embodiment, the electroless plating may be performed under alkaline conditions. It will be understood that an acid or base may be used to control the pH range. In some embodiments, the pH range may be in the range from about 4.5 to about 10. The pH may be in the range of from about 4.7 to about 9.8, about 4.9 to about 9.5, about 5.1 to about 9.3, or about 5.3 to about 9.2. The pH may be at least 4.5, at least 4.6, at least 4.7, at least 4.8, at least 4.9, at least 5.0, at least 5.0, at least 5.2, at least 5.3, at least 5.4, or at least 5.5. The pH may be less than 10, less than 9.9, less than 9.8, less than 9.7, less than 9.6, less than 9.5, less than 9.4, less than 9.3, less than 9.2, less than 9.1, less than 9.0, less than 8.9, or less than 8.8. The pH may be in a range provided by any lower and/or upper limit as previously described.

Once the electroplating reaction commences, the deposition of the ionic compound (i.e. ionic shell) may become auto-catalytic. In an embodiment, the thickness of the ionic shell may be controlled by limiting the concentration of the ions of the in solution and/or the duration of the electroless deposition procedure. In some embodiments or examples, further advantages are provided by the ionic shell being an inorganic calcium phosphate shell, which can provide further effective auto-catalyses.

In some embodiments or examples, the ratio of calcium ions to phosphate ions may be between about 20:1 to about 0.1:1. The ratio of calcium ions to phosphate ions may be in the range of from about 15:1 to about 0.2:1, about 10:1 to about 0.4:1, about 5:1 to about 0.5:1, or about 2:1 to about 1:1. The ratio of calcium ions to phosphate ions may be at least 0.1:1, at least 0.2:1, at least 0.4:1, at least 0.5:1, at least 1:1, at least 2:1, or at least 4:1. The ratio of calcium ions to phosphate ions may be less than 15:1, less than 10:1, less than 4:1, less than 2:1, less than 1:1, less than 0.5:1, or less than 0.2:1. The ratio of calcium ions to phosphate ions may be in a range provided by any lower and/or upper limit as previously described.

Suitable techniques for conducting the electroless plating procedure are described, for example, in the following documents: Basarir et al., ACS Applied Materials & Interfaces, 2012, 4(3), 1324-1329; Blake et al., Langmuir, 2010, 26(3), 1533-1538; Chen et al., Journal of Physical Chemistry C, 2008, 112(24), 8870-8874; Fujiwara et al., Journal of the Electrochemical Society, 2010, 157(4), pp. D211-D216; Guo et al., Journal of Applied Polymer Science, 2013, 127(5), 4186-4193; Haag et al., Surface and Coatings Technology, 2006, 201(6), 2166-2173; Horiuchi et al., Surface & Coatings Technology, 2010, 204(23), 3811-3817; Ko et al., Journal of the Electrochemical Society, 2010, 157(1), pp. D46-D49; Lin et al., International Journal of Hydrogen Energy, 2010, 35(14), 7555-7562; Liu et al., Langmuir, 2005, 21(5), 1683-1686; Ma et al., Applied Surface Science, 2012, 258(19), 7774-7780; Miyoshi et al., Colloids and Surfaces A: Physicochemical and Engineering Aspects, 2008, 321(1-3), 238-243; Moon et al., 2008, Ultramicroscopy, 108(10), 1307-1310; Wu et al., Journal of Colloid and Interface Science, 2009, 330(2), 359-366; Ye et al., Materials Letters, 2008, 62(4-5), 666-669; and Zhu et al., Surface and Coatings Technology, 2011, 205(8-9), 2985-2988.

In some embodiments or examples, the ions of the ionic compound may be present in the solution at a concentration of from 0.05 to 2000 mM. The concentration may be in a range of about 0.1 to 1500 mM, 0.5 to 1000 mM, 1.0 to 800 mM, or 10 to 500 mM. The concentration may be at least 5 mM, at least 10 mM, at least 15 mM, at least 20 mM, at least 25 mM, at least 30 mM, at least 45 mM, at least 60 mM. The concentration may be less than 800 mM, less than 500 mM, less than 250 mM, 230 mM, less than 225 mM, less than 200 mM, less than 150 mM, less than 100 mM, or less than 50 mM. The concentration may be in a range provided by any lower and/or upper limit as previously described. In an example, calcium chloride may be provided in a concentration range of 10 to 500 mM, 45 to 225 mM, or 60 to 100 mM, and hypophosphate may be provided in a concentration range of 10 to 500 mM, 25 to 230 mM, or 30 to 100 mM.

In some embodiments or examples, the electroless plating process may be performed at temperature in a range from between 10° C. to 100° C. The temperature may be in the range of from about 15° C. to about 95° C., about 20° C. to about 90° C., about 25° C. to about 85° C., about 30° C. to about 80° C., about 35° C. to about 75° C., about 40° C. to about 70° C., or about 45° C. to about 65° C. The temperature may be at least 15° C., at least 20° C., at least 25° C., at least 30° C., at least 35° C., at least 40° C., at least 45° C., at least 50° C., at least 55° C., at least 60° C., or at least 65° C. The temperature may be less than 90° C., less than 85° C., less than 80° C., less than 75° C., less than 70° C., less than 75° C., less than 70° C., less than 65° C., less than 60° C., or less than 55° C. The temperature may be in a range provided by any lower and/or upper limit as previously described.

### Impermeability and Leakage Tests

The microcapsules may be characterised in terms of their permeability. The permeability may be tested using the Ethanol Stability test where a known volume of microcapsules can be isolated and dispersed in an aqueous solution comprising a 1:4 solution of water to absolute ethanol. The dispersion can be heated to 40° C. After 7 days at 40° C., the microcapsules can be isolated from the aqueous solution using centrifugation at 7000 rpm for 1 minute. The aqueous solution can then be subjected to analysis using gas chromatography to determine the content of the fluid core material that has leached from the microcapsules. To confirm the presence of the fluid core material within the microcapsules, a known sample of microcapsules can be crushed between two glass slides and washed into a vial with 5 ml ethanol. The microcapsules can be isolated from the aqueous solution using centrifugation at 7000 rpm for 1 minute. The aqueous solution can then be subjected to analysis using gas chromatography or liquid chromatography-ultraviolet spectroscopy-mass spectroscopy (LC-UV-MS) to determine the content of the fluid core material that has leached from the microcapsules.

The microcapsules may also be characterised in terms of efficiency. To determine the efficiency of the outer shell of the microcapsule, NMR may be utilized to determine leakage of the fluid core. For example, ¹⁹F NMR may be used to determine leakage of a perfluorooctyl bromide liquid core. In an embodiment or example, the microcapsule can be left to stand for two weeks in a solvent (e.g. chloroform-D with perfluorobenzoic acid (PFBA)) solution to assess the ability of the outer shell to prevent leakage. It will be appreciated that the any imperfections within the outer shell of the microcapsule can allow for dissolution of the polymer inner coating in the solvent and subsequent loss of the liquid from the core, which can be detected by NMR from characteristic peaks of the fluid core. In other words, the release of the fluid core from the microcapsule can be detected on the NMR spectrum. After two weeks in the solvent environment the fluid core can be was detected and compared with the internal standard concentration.

Advantageously, the microcapsules can be delivered in a targeted manner or in response to a specific trigger. According to at least some embodiments or examples as described herein, the microcapsules can provide a capsule that is substantially impermeable and can be advantageously suitable for use in various applications. The microcapsule can be impermeable to low molecular weight volatile molecules encapsulated within it thereby preventing release. The inventors have surprisingly found that depositing an ionic shell on a microcapsule, for example depositing an inorganic calcium phosphate shell on a microcapsule, can provide a substantially impermeable microcapsule suitable for a number of applications, including but not limited to, drug delivery, personal care products, agricultural products and food products. In some embodiments or examples, the ionic shell may be substantially impermeable to low molecular weight or volatile “active agent” molecules, for example molecules having a molecular weight of less than about 1000 g·mol⁻¹, 900 g·mol⁻¹, 800 g·mol⁻¹, 700 g·mol⁻¹, 600 g·mol⁻¹, 500 g·mol⁻¹, 400 g·mol⁻¹, 300 g·mol⁻¹, or 200 g·mol⁻¹. In an example, the microcapsule may be impermeable to molecules smaller than 500 g·mol⁻¹. In another example, the microcapsules can retain low molecular weight active agents present in the fluid core of the microcapsules for up to about 12 hours, 24 hours, 1 day, 2 days, 3 days, 4 days, 5 days, 6 days, 7 days, 2 weeks, 3 weeks, 4 weeks, or 2 months. The impermeability or retention of active agent in the fluid core may be measured by placing the prepared microcapsules into a solution (e.g. chloroform-D) for predetermined time, such as 1 week, and measuring the amount of active agent released into the solution. In an embodiment or example, the microcapsules may retain at least 50% by weight of the inner fluid core. The retention of active agent within the microcapsule as a weight % of active agent may be at least about 50%, 60%, 70%, 80%, 85%, 90%, 95%, 98%, 99%, 99.5%, 99.8%, or 99.9%. The retention of the active agent within the microcapsule as a weight % of active agent may be less than, 99.99%, 99.9%, 99.8%, 99.5%, 99%, 98%, 95%, 90%, 85%, 75%, or 55%. The retention of the active agent within the microcapsule may be in a range provided by any lower and/or upper limit as previously described.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

### EXAMPLES

The present disclosure is further described by the following examples. It is to be understood that the following description is for the purpose of describing particular examples only and is not intended to be limiting with respect to the above description.

### Example 1a: Preparation of Polymeric Coated Liquid Core Comprising Platinum Nanoparticles

Platinum nanoparticles (Pt-NP) can act as the catalyst for the deposition of the inorganic material layer (e.g. calcium phosphate—CaP), which can be incorporated or embedded into the liquid core or inner coating encapsulating the liquid core.

Firstly, polyvinylpyrrolidone (PVP)-stabilised platinum nanoparticles (PVP—Pt-NP) were synthesised. H₂PtCl₆(0.115 g, 2.2 mM) was dissolved in 100 mL of PVP solution (1.15 μM). In a 250 mL conical flask the H₂PtCl₆ solution was stirred vigorously and NaBH₄ (0.4 mL, 0.5 M) was rapidly injected into the flask. The yellow solution immediately turned dark brown, and after two minutes of vigorous stirring was left to stand for at least 12 hours to form a dark brown nanoparticle dispersion of polymer stabilised platinum nanoparticles (PVP-Pt-NP).

To form an inner coated liquid core, an inner coating material of poly(lactic-co-glycolic acid) (PLGA) (0.1 g) was dissolved in DCM (4 mL) before a liquid core composition comprising perfluorooctyl bromide (PFOB) (60 μL), or hexyl salicylate (60 μL) was added and mixed until a single oil phase was formed. PVP (2.0 g) was dissolved into 100 mL ultrapure water (18.2 MΩ cm at 25° C.). To form the aqueous phase, the PVP solution (15 mL) and PVP-Pt-NP (5 mL) were added carefully on top of the oil phase. This was homogenised (IKA T25 Ultra-Turrax) at 17500 rpm for 2 min, over ice to form an oil-in-water emulsion. The emulsion was then magnetically stirred at 300 rpm for 24 hours to allow for complete evaporation of the DCM and precipitation of a plurality of polymer coated microcapsules (also referred to below as “PLGA capsules” or “PVP-Pt stabilized capsules”). The polymer coated microcapsules were washed twice via centrifugation at 2000 rpm for 10 minutes at 10° C. followed by redispersion in 25 mL ultrapure water.

### Preparation of Microcapsules Comprising a Calcium Phosphate Ionic Shell:

For electroless deposition of CaP onto PLGA microcapsules, 1 mL of Pt-PVP stabilized microcapsules were added to a plating solution of calcium chloride (0.33 mL, 96.6 mM (1.05 g in 100 mL H₂O), optionally sodium fluoride (0.33 mL, 1.19 mM (0.5 g in 100 mL H₂O)), sodium hypophosphite (0.33 mL, 47.187 mM (0.5 g in 100 mL H₂O), and succinic acid (0.33 mL, 0.59 mM (0.7 g in 100 mL H₂O)). This was stirred magnetically at 400 rpm for 15 min at 60° C. This was then washed via centrifugation at 2000 rpm for 10 min at 10° C. and redispersed in 3 mL Milli-Q water to provide a plurality of microcapsules having an outer ionic shell of calcium phosphate encapsulating an inner coated liquid core comprising platinum nanoparticles (also referred to herein as “CaP coated microcapsule”).

### Example 1b: Characterisation

The size distribution of the CaP coated and uncoated polymeric PLGA microcapsules was measured by dynamic light scattering (DLS—Malvern Zetasizer)) and transmission electron microscopy (TEM) with image analysis performed in ImageJ to determine the CaP shell thickness. Elemental composition analysis and elemental mapping were investigated using TEM with energy dispersive X-ray (EDX) (Hitachi HT7700 TEM at 100 kV). The TEM samples were prepared by placing two droplets of dispersed experimental solution onto a carbon-coated 300 mesh copper grid. Images were analysed using ImageJ to quantify size distribution of the capsules.

The morphology of the ionic shell microcapsules was analysed using a JEOL JCM-5000 neoScope scanning electron microscope and JEOL JSM-7100F scanning electron microscope (JEOL, USA). All samples were sputter coated with 3 nm iridium prior to imaging. Microcapsule permeability was assessed by nuclear magnetic resonance (NMR) using ¹⁹F NMR (Bruker AVIII 400 MHz spectrometer) using deuterated chloroform as the solvent with pentafluorobenzoic acid (pFBA, 10 mg/mL) as the internal standard. Leakage tests were conducted for two weeks at 40° C. and six weeks at room temperature.

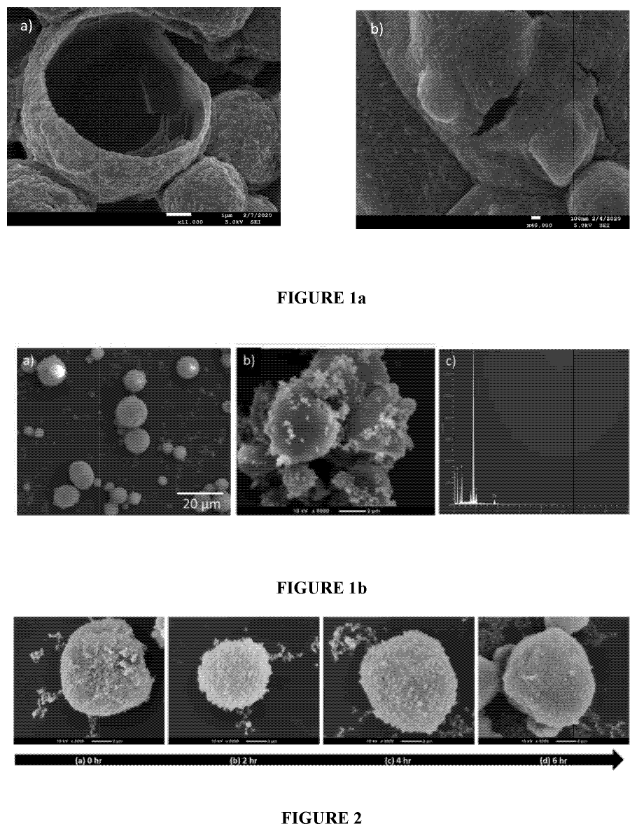

To investigate the formation of calcium phosphate shells a range of calcium chloride concentrations were investigated. Further improved calcium phosphate coatings were observed in reactions where CaCl₂ concentration was varied between 56 and 192 mM, particularly when NaH₂PO₂.H₂O concentrations were kept constant at about 47 mM. This suggests the reaction is highly sensitive to the amount of hypophosphite used. FIG. 1a shows CaP coated PLGA microcapsules having two different shell thicknesses, achieved with 96 mM CaCl₂ and 192 mM CaCl₂ reagent concentration, respectively.

FIG. 1b shows the morphology of the calcium phosphate shell under different reaction conditions for Example 1. Elemental analysis was conducted on the sample shown in FIG. 1a and the EDX spectrum in FIG. 1c confirms the presence of calcium and phosphate in the microcapsule shell. This confirms platinum nanoparticles to be an efficient catalyst for the electroless deposition of calcium phosphate.

To demonstrate the mechanism of calcium phosphate growth as an impermeable shell on the PLGA capsules in Example 1, the time allowed for electroless deposition of calcium phosphate was varied. The sample from FIG. 1a with a molar ratio of CaCl₂:NaH₂PO₂ of 2.1:1 was used here. The samples shown in FIG. 2 were left to stand at room temperature for varying time periods after the initial electroless plating reaction at 60° C. for 15 minutes. This suggests that the CaP ionic shell auto-catalyses further deposition over time. After 6 hours of standing at room temperature, the capsules appeared to be successfully surrounded with a thicker calcium phosphate film. The same trend was observed when the capsules were left to stand overnight before washing via centrifugation. The CaP coating remained visually intact, fully surrounding the polymeric biomaterial with an average inorganic shell thickness of 32 nm, based on the change in size as measured using dynamic light scattering before and after coating. The decomposition and degradation of polymeric shell are significantly reduced at this low-temperature synthesis route, making it economically feasible to produce, rather than sintering at higher temperatures which can be expensive to maintain.

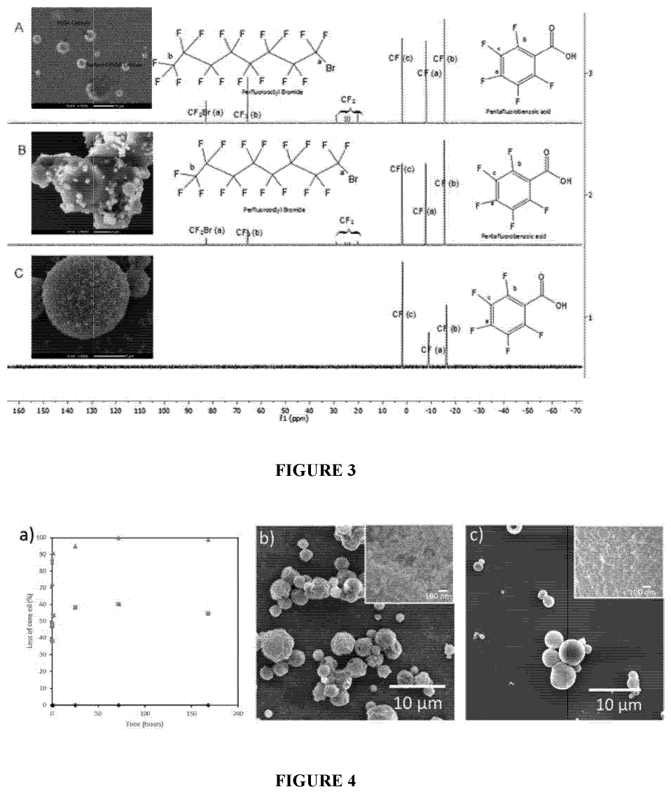

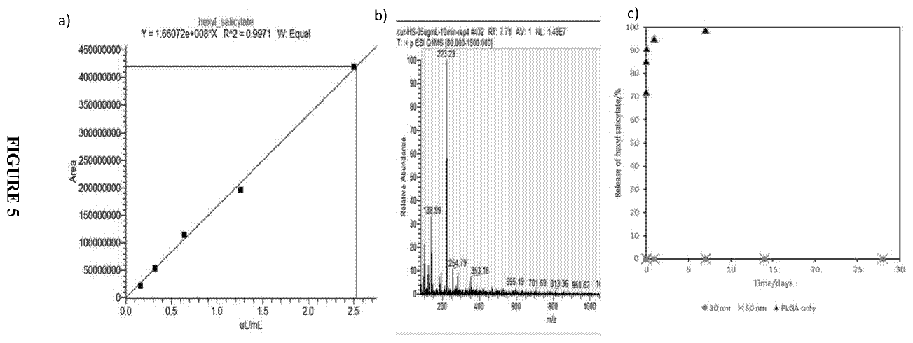

To determine the efficiency of the protective calcium phosphate shell, ¹⁹F NMR was used to check for any potential leakage of the PFOB core in Example 1. The capsules were left to stand for two weeks in chloroform-D with perfluorobenzoic acid (PFBA) solution to assess the ability of the calcium phosphate coating to prevent leakage. PLGA is highly soluble in chloroform, and thus full release of the PFOB from within the capsule core will be detected on the ¹⁹F NMR spectrum, as seen in FIG. 3a which shows the spectrum obtained for PLGA capsules with no additional coating. Any imperfections within the CaP shells will allow for dissolution of the polymer shell in the solvent and subsequent loss of PFOB from the core, which can be detected from the characteristic peaks of PFOB. A partially coated sample, FIG. 1b, was used to obtain the spectrum in FIG. 3b. The SEM image showed that the PLGA capsules were not thoroughly coated by the protective barrier of calcium phosphate. Consequently, after two weeks in the solvent environment PFOB was detected and by comparison with the internal standard concentration, a leakage of 60% was detected. For the apparent fully coated sample in FIG. 1a, a 100% retention efficiency is seen of PFOB in FIG. 3c, as evidenced by the lack of chemical shifts associated with PFOB in the NMR spectrum. Only the chemical shifts of pentafluorobenzoic acid were detected, confirming an impermeable shell of calcium phosphate had been deposited on the PLGA capsules. Further analyses were done on these capsules, investigating the time frame for an efficient core retention. The obtained results showed that the retention efficiency was as high as 95% suggesting that the capsules can withstand a harsh, unfavourable environment for at least six weeks.

To determine the permeability of the protective calcium phosphate shell, the Ethanol Stability Test was conducted. The microcapsules (1 mL) were dispersed in 4 mL absolute ethanol, and mixed by carousel at room temperature for 7 days. At desired timepoints ranging from 2 minutes to 28 days, a known volume (250 uL) of microcapsule dispersion was removed from the sample for the aqueous phase to be tested for the presence of hexyl salicylate using gas chromatography (Shimazdu GC2010Plus). The microcapsules were isolated from the aqueous solution using centrifugation at 7000 rpm for 1 minute. The aqueous solution was then analysed using gas chromatography to determine the content of the liquid core material that has leached from the microcapsules. A HP-Ultra-1 column (25 m, 0.2 mm diameter, 0.33 μm film) was used. The injection and detection ports were set to 300° C. and a 2 μL injection volume was used. The gas flow was 1.74 mL/minute and the temperature was ramped from 50 to 300° C. at a rate of 20° C. per minute. This result was confirmed using liquid chromatography-ultraviolet spectroscopy-mass spectrometry (LC-UV-MS) (Thermo Quantum Ultra QqQMS with Dionex U3000 with VWD detector, Thermo Scientific, USA). A reverse phase C18 column (50 mm×2.1 mm) was used with 10 μL sample injection with 80% acetonitrile (aq) and 18 MΩ purified water, both with 0.2% formic acid, as the solvent and a flow rate of 200 μL/min, and UV detection at 214, 250 and 425 nm. The ratio of solvents was varied as follows: 0-1 minute 50:50, ramped to 90:10 by 6 minutes, and 100% acetonitrile at 6.6-8.8 minutes before being reduced to 50:50 until 10 minutes. The concentration of hexyl salicylate release was calculated from a calibration curve of peak area as a function of known hexyl salicylate concentrations. FIG. 5 shows release data of hexyl salicylate from PLGA only microcapsules and from two samples of CaP coated microcapsules shown in example 1a, prepared with 96 mM and 192 mM CaCl₂ reagent concentration. The release data over 28 days advantageously shows that the liquid core, hexyl salicylate, had not leached from the CaP coated microcapsules.

### Example 2a: Preparation of Liquid Core Comprising Platinum Nanoparticles at the Interface (Pickering Emulsion)

Polyvinylpyrrolidone (PVP)-stabilised platinum nanoparticles (PVP-Pt-NP) were synthesised. H₂PtCl₆ (0.23 g) was dissolved in 100 mL of PVP solution (0.00625 wt %). In a 250 mL conical flask the H₂PtCl₆ solution was stirred vigorously and NaBH₄ (2.0 mL, 1.1 M) was rapidly injected into the flask. The yellow solution immediately turned dark brown, and after two minutes of vigorous stirring was left to stand for at least 12 hours to form a dark brown nanoparticle dispersion of polymer stabilised platinum nanoparticles (PVP-Pt-NP).

To form an liquid core, an aqueous phase of PVP-Pt-NP (0.45 mL) and 0.45 ml of ultrapure water was added carefully on top of an oil phase (toluene or miglyol). This was homogenised (IKA T25 Ultra-Turrax) at 17500 rpm for 2 min, over ice to form an oil-in-water emulsion. The emulsion was then magnetically stirred at 300 rpm for 24 hours to allow precipitation of a plurality of. The microcapsules were washed twice via centrifugation at 2000 rpm for 10 minutes at 10° C. followed by redispersion in 25 mL ultrapure water.

### Preparation of Microcapsules Comprising a Calcium Phosphate Ionic Shell:

For electroless deposition of CaP onto the microcapsules, 0.5 mL of Pt-PVP stabilized microcapsules were added to a plating solution of calcium chloride (1.0 mL, 192 mM, optionally sodium fluoride (1.0 mL, 1.19 mM), sodium hypophosphite (1.0 mL, 47 mM), and succinic acid (1.0 mL, 0.59 mM). This was stirred magnetically at 400 rpm for 15 min at 60° C. This was then washed via centrifugation at 2000 rpm for 10 min at 10° C. and redispersed in 3 mL ultrapure water to provide a plurality of microcapsules having an outer ionic shell of calcium phosphate encapsulating a liquid core comprising platinum.

### Example 2b: Characterisation

CaP coated microcapsule emulsions were observed using scanning electron microscopy, sputter coated with 15 nm carbon. The CaP coated microcapsules ruptured upon drying and evidence of oil was visible surrounding the fragmented CaP ionic shells. Topography consistent with a CaP ionic shell was observed and elemental analysis confirmed that CaP was present.

### Example 3a: Preparation of Gel Core Comprising Platinum Nanoparticles at the Interface of a Hydrogel

Toluene (1 mL) was emulsified with 3.7 mL low viscosity sodium alginate (1.5 wt % in water, viscosity at 2% approx. 250 cps) and 0.3 mL poly(vinyl pyrrolidine) (1 wt % solution) using an ultraturrax for 2 minutes at 20000 rpm. Using a syringe with 27 G needle, this solution was added dropwise into a stirred beaker of calcium chloride (100 mM, 10 mL). Gel beads formed immediately upon contact with the calcium chloride solution, and were left to stir, sealed for 1 hour. 10 beads were removed and added to 0.5 mL PVP-Pt-NP (synthesised as described in Example 1a) and 4 mL water, and stirred for 4 hours for adsorption of the nanoparticles to the bead interface. The aqueous phase was removed and replaced with 1 mL water.

### Preparation of Microcapsules Comprising a Calcium Phosphate Ionic Shell:

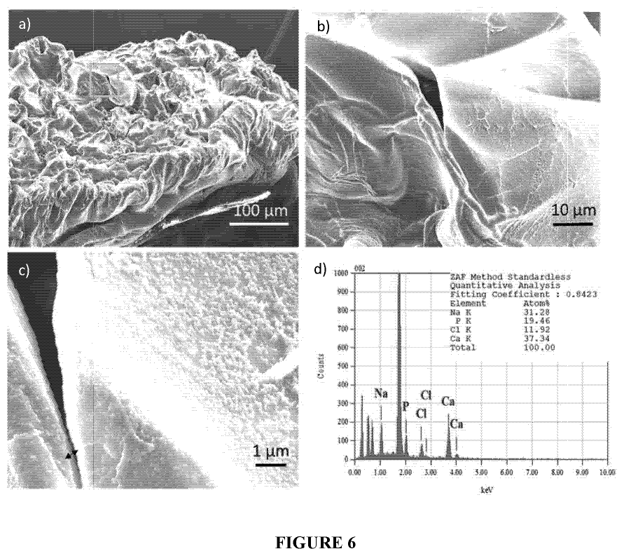

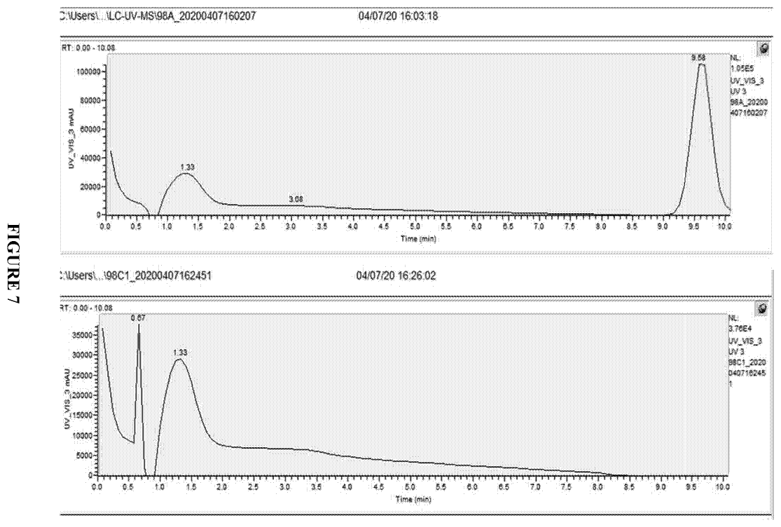

For electroless deposition of CaP onto the microcapsules 5 beads were added to a calcium phosphate plating solution, consisting of calcium chloride (1.0 mL, 192 mM, optionally sodium fluoride (1.0 mL, 192 mM), optionally sodium fluoride (1.0 mL, 1.19 mM), sodium hypophosphite (1.0 mL, 47 mM), and succinic acid (1.0 mL, 0.59 mM). This was stirred magnetically at 400 rpm for 15 min at 60° C. This was then washed by removal of the aqueous phase and redispersion in 3 mL ultrapure water to provide a plurality of microcapsules having an outer ionic shell of calcium phosphate encapsulating a gel core comprising platinum nanoparticles. The scanning electron micrographs shown in FIG. 6 advantageously show that complete calcium phosphate shell has been deposited onto the calcium alginate microcapsule beads with PVP-PT-NPs adsorbed. The elemental analysis also provided in FIG. 6 confirms the presence of Ca and P elements

### Example 3b: Characterisation

CaP coated microcapsule beads were observed using scanning electron microscopy, sputter coated with 15 nm carbon. Topography consistent with a CaP ionic shell was observed on the beads and elemental analysis confirmed that CaP was present. Retention of toluene from the beads was confirmed using UV spectroscopy at a wavelength of 265 nm (FIG. 7). A sample of CaP coated beads containing 14 uL/mL⁻¹ of toluene was added to 4:1 ethanol:water and left stirring for 7 days before the supernatant was removed for UV analysis. This was compared to uncoated beads containing 14 uL/mL⁻¹ of toluene. The absorption corresponding to toluene was detected by LC-UV-MS at a retention time of 9.3 minutes in the uncoated beads. No signal was detected for the CaP coated microcapsule beads, confirming no toluene was released into the supernatant.

1. A microcapsule comprising an ionic shell encapsulating a fluid core, wherein the fluid core comprises platinum nanoparticles.

2. The microcapsule of claim 1, wherein the ionic shell comprises an alkaline earth metal.

3. The microcapsule of claim 2, wherein the alkaline earth metal is selected from beryllium (Be), magnesium (Mg), calcium (Ca), strontium (Sr), barium (Ba), or combinations thereof.

4. The microcapsule of any one of the preceding claims, wherein the ionic shell comprises or consists of one or more ionic compounds selected from phosphates, sulphates, nitrates, silicates, carbonates, or combinations thereof.

5. The microcapsule of any one of the preceding claims, wherein the ionic shell comprises or consists of one or more ionic compounds selected from beryllium phosphate, beryllium sulphate, beryllium nitrate, beryllium silicate, beryllium carbonate, magnesium phosphate, magnesium sulphate, magnesium nitrate, magnesium silicate, magnesium carbonate, calcium phosphate, calcium sulphate, calcium nitrate, calcium silicate, calcium carbonate, strontium phosphate, strontium sulphate, strontium nitrate, strontium silicate, strontium carbonate, barium phosphate, barium sulphate, barium nitrate, barium silicate, barium carbonate, or combinations thereof.

6. The microcapsule of any one of the preceding claims, wherein the ionic shell is an inorganic calcium phosphate shell comprising or consisting of one or more calcium phosphate compounds selected from monocalcium phosphate, dicalcium phosphate, tricalcium phosphate, octacalcium phosphate, dicalcium diphosphate, calcium triphosphate, calcium hydroxide phosphate, or combinations thereof.

7. The microcapsule of any one of the preceding claims, wherein the ionic shell is formed around the fluid core.

8. The microcapsule of any one of the preceding claims, wherein the fluid core is a liquid core or a gel core.

9. The microcapsule of any one of the preceding claims, wherein the platinum nanoparticles are polymer stabilised platinum nanoparticles.

10. The microcapsule of any one of the preceding claims, wherein the platinum nanoparticles comprise or consist of one or more platinum compounds selected from PtCl₂, H₂PtCl₆, K₂PtCl₆, Na₂PtCl₆, K₂PtCl₄, or combinations thereof.

11. The microcapsule of any one of the preceding claims, wherein the diameter of the platinum nanoparticles is between about 0.1 nm and 200 nm.

12. The microcapsule of any one of the preceding claims, wherein the ionic shell has a thickness of between about 1 nm to about 1000 nm.

13. The microcapsule of any one of the preceding claims, wherein the ionic shell has a thickness of between about 20 nm to 150 nm.

14. The microcapsule of any one the preceding claims, wherein the ionic shell is impermeable to molecules smaller than 500 g·mol⁻¹.

15. The microcapsule of any one of the preceding claims, wherein the fluid core comprises a fluid carrier comprising one or more active agents and the platinum nanoparticles.

16. The microcapsule of any one of the preceding claims, wherein the fluid core further comprises one or more additives selected from an oil carrier, an aqueous carrier, a solid, a water/oil emulsion, and contrast agents.

17. The microcapsule of any one of the preceding claims, wherein the fluid core comprises between 45% to about 99.9% by weight of the coated microcapsule.

18. The microcapsule of any one of the preceding claims, wherein the active agent in the fluid core is selected from pharmaceuticals, nutraceuticals, pesticides, insecticides, fertilizers, herbicides, perfumes, brighteners, insect repellents, silicones, waxes, flavours, vitamins, fabric softening agents, depilatories, skin care agents, enzymes, probiotics, dye polymer conjugate, perfume delivery system, sensates, attractants, anti-bacterial agents, dyes, pigments, bleaches, flavourants, sweeteners, waxes, UV blockers/absorbers, or combinations thereof.

19. The microcapsule of any one of the preceding claims, wherein the fluid core further comprises an inner coating encapsulating the fluid core, and wherein the ionic shell encapsulates the inner coating.

20. The microcapsule of claim 19, wherein the inner coating is a polymeric shell.

21. The microcapsule of claim 20, wherein the polymeric shell is selected from a synthetic polymer or a naturally-occurring polymer.

22. The microcapsule of claim 21, wherein the synthetic polymer is selected from nylon, polyethylenes, polyamides, polystyrenes, polyisoprenes, polycarbonates, polyesters, polyureas, polyurethanes, polyolefins, polysaccharides, epoxy resins, vinyl polymers, polyacrylates, or combinations thereof.

23. The coated microcapsule of any one of claims 20 to 22, wherein the polymeric shell comprises a thermoplastic polymer.

24. The microcapsule of claim 21, wherein the naturally-occurring polymer is selected from silk, wool, gelatin, cellulose, alginate, proteins, or combinations thereof.

25. The microcapsule of any one of claims 20 to 24, wherein the polymeric shell comprises a homopolymer or a copolymer.

26. The microcapsule of any one of claims 20 to 25, wherein the polymeric shell comprises a biodegradable polymer.

27. The microcapsule of claim 20, wherein the polymeric shell comprises a polyester.

28. The microcapsule of claim 27, wherein the polyester is selected from polyglycolic acid, polylactic acid, polycaprolactone, polyhydroxyalkanoate, polyhydroxybutyrate, polyethylene adipate, polybutylene succinate, polytrimethylcarbonate-co-lactide, or combinations thereof.

29. The microcapsule of claim 28, wherein the polyester is a copolymer of polylactic acid and polyglycolic acid.

30. The microcapsule of claim 20, wherein the polymeric shell comprises a polyacrylate.

31. The microcapsule of claim 30, wherein the polyacrylate is selected from poly(methyl methacrylate) or poly(ethyl methacrylate).

32. The microcapsule of any one of claims 19 to 31, wherein the inner coating has a thickness of between about 10 μm to about 5000 μm.

33. The microcapsule of any one of claims 19 to 32, wherein the ratio by weight of the fluid core to inner coating is between about 6:1 to 1:1.

34. The microcapsule of any one of the preceding claims, wherein the platinum nanoparticles are stabilised using polyvinylpyrrolidone, polyvinyl alcohol, polyethyleneimine.

35. The microcapsule of any one of the preceding claims, wherein the fluid core and/or inner coating comprise platinum nanoparticles for catalysing an electroless plating deposition of the ionic shell.

36. The microcapsule of claim 35, wherein the platinum nanoparticles are present in the fluid core at the surface of the fluid core as a Pickering stabiliser.

37. The microcapsule of claim 35, wherein the platinum nanoparticles are present in the inner coating as a Pickering stabiliser.

38. The microcapsule of any one of the preceding claims, wherein the diameter of the microcapsule is between about 0.05 μm to about 1000 μm.

39. The microcapsule of any one of the preceding claims, wherein the ionic shell further comprises one or more trace elements selected from titanium, iron, silver, copper, gold, zinc, manganese, strontium, lithium, silicon, fluorine, sodium, barium, and magnesium, forming an ionic shell composite.

40. A composition comprising a plurality of microcapsules according to any one of the preceding claims.

41. A process for preparing a microcapsule, the process comprising providing a fluid core comprising platinum nanoparticles, and encapsulating the fluid core with an ionic shell by electroless plating of the ionic shell onto the fluid core.

42. The process of claim 41, wherein the platinum nanoparticles is embedded within the fluid core prior to deposition of the ionic shell.

43. The process of claim 41 or claim 42, wherein the fluid core is a liquid or a gel.

44. The process of any one of claims 41 to 43, further comprising encapsulating the fluid core with an inner polymeric coating using an emulsification process, and then encapsulating the inner polymeric coating with an ionic shell.

45. The process of claim 44, wherein the platinum nanoparticles are adsorbed within the inner polymeric coating to form a discontinuous layer during the emulsification process.

46. The process of any one of claims 41 to 45, wherein the ionic shell further comprises one or more trace elements selected from titanium, iron, silver, copper, gold, zinc, manganese, strontium, lithium, silicon, fluorine, sodium, barium, and magnesium, forming an ionic shell composite.

47. Use of a microcapsule of any one of claims 1 to 39, or composition comprising a plurality of microcapsules of claim 40, as a drug delivery vehicle or carrier in controlled release of the fluid core comprising an active agent.

48. Use according to claim 47, wherein the microcapsule is used as an implant within a subject for controlled release of the active agent to the subject.

49. Use according to claim 48, wherein the controlled release is for providing systemically administered doses.

50. Use of a microcapsule of any one of claims 1 to 39, or composition comprising a plurality of microcapsules of claim 40, in personal care products.

51. Use of a microcapsule of any one of claims 1 to 39, or composition comprising a plurality of microcapsules of claim 40, in agricultural products.

52. Use according to any one of claims 47 to 51, wherein the release of the fluid core comprising the active agent from the microcapsule is activated by ultrasound, degradation, or mechanical fracture.
